(19) United States
(12) Patent Application Publication
(10) Pub. No.: US 2023/0317164 A1
(43) Pub. Date: Oct. 5, 2023

(54) SYSTEMS AND METHODS FOR WRITING BY SEQUENCING OF NUCLEIC ACIDS

(71) Applicant: CATALOG TECHNOLOGIES, INC., Charlestown, MA (US)

(72) Inventors: Nathaniel Roquet, Charlestown, MA (US); Swapnil P. Bhatia, Charlestown, MA (US); Devin Leake, Charlestown, MA (US)

(21) Appl. No.: 18/024,909

(22) PCT Filed: Sep. 7, 2021
(86) PCT No.: PCT/US2021/049289

§ 371 Date: Mar. 6, 2023

Related U.S. Application Data
(60) Provisional application No. 63/075,622, filed on Sep. 8, 2020.

(51) Int. Cl. G11C 13/02; C12Q 1/6855; C12Q 1/6869; G16B 50/30; G06N 3/123

(52) U.S. Cl. CPC G11C 13/02; C12Q 1/6855; C12Q 1/6869; G16B 50/30; G06N 3/123

(57) ABSTRACT

The systems, devices, and methods described herein provide nucleic acid digital data storage encoding and retrieving methods that are less costly and easier to commercially implement than existing methods. The systems, devices, and methods described herein provide scalable methods for writing data to and reading data from nucleic acid molecules. The present disclosure covers five primary areas of interest: (1) writing digital information into nucleic acid molecules, (2) accurately and quickly reading information stored in nucleic acid molecules, (3) partitioning data to efficiently encode data in nucleic acid molecules, (4) error protection and correction when encoding data in nucleic acid molecules, and (5) data structures to provide efficient access to information stored in nucleic acid molecules.

### REFERENCE TO RELATED APPLICATIONS

This application claims priority to and the benefit of U.S. Provisional Patent Application No. 63/075,622, filed on Sep. 8, 2020. The entire contents of the above-referenced application is incorporated herein by reference.

### BACKGROUND

Nucleic acid digital data storage is a stable approach for encoding and storing information for long periods of time, with data stored at higher densities than magnetic tape or hard drive storage systems. Current methods for nucleic acid digital data storage rely on encoding the digital information (e.g., binary code) into base-by-base nucleic acids sequences, such that the base-to-base relationship in the sequence directly translates into the digital information (e.g., binary code) using multi-step chemistry. Sequencing of digital data stored in base-by-base sequences that can be read into bit-streams or bytes of digitally encoded information can be error prone and costly to encode since the cost of de novo base-by-base nucleic acid synthesis can be expensive.

Reading times and costs may be prohibitive for data written with base-by-base synthesis. Information stored at a high density (in terms of bits-per-base) may need high accuracy and high-resolution sequencing to read back. Indeed, data stored at or near the theoretical maximum of 2 bits/base has similar stringency requirements for sequencing as does genomic information. This leaves little room for innovation over state-of-the-art sequencers intended for genomic applications. For reference, approximately 450B reads would need to be processed to recover a full TB of data, which can cost millions of dollars and thousands of hours to process. One method of sequencing is a nanopore sequencing. A key hurdle for nanopore sequencing has been achieving slow enough DNA translocation and narrow enough pores to sequence individual DNA bases.

### SUMMARY

The systems, devices, and methods described herein provide nucleic acid digital data storage encoding and retrieving methods that are less costly and easier to commercially implement than existing methods. The systems, devices, and methods described herein provide scalable methods for writing data to and reading data from nucleic acid molecules. The present disclosure covers five primary areas of interest: (1) writing digital information into nucleic acid molecules, (2) accurately and quickly reading information stored in nucleic acid molecules, (3) partitioning data to efficiently encode data in nucleic acid molecules, (4) error protection and correction when encoding data in nucleic acid molecules, and (5) data structures to provide efficient access to information stored in nucleic acid molecules.

In an aspect, a method for writing digital information into nucleic acid molecules, includes mapping the digital information to a target set of identifier nucleic acid sequences. The method includes obtaining a plurality of identifier nucleic acid molecules. The method includes sequencing an identifier nucleic acid molecule of said plurality of identifier nucleic acid molecules with a nanopore system. The method includes accepting or rejecting the identifier nucleic acid molecule into a destination chamber based on whether or not the identifier nucleic acid molecule corresponds to an identifier nucleic acid sequence of the target set.

In some implementations, said mapping includes using a codebook that maps a word to a codeword. In some implementations, at least one identifier nucleic acid sequence corresponds to a bit in the codeword.

In some implementations, if said bit has a bit-value of 1, said bit is represented by a presence of the at least one corresponding identifier nucleic acid sequence in the target set, and if the bit has a bit-value of 0, said bit is represented by an absence of any corresponding identifier nucleic acid sequences in the target set.

In some implementations, said plurality of identifier nucleic acid molecules is obtained by assembling multiple component nucleic acid molecules using a product scheme. In some implementations, the product scheme defines a set of M layers, each layer including a set of components. In some implementations, each identifier nucleic acid molecule contains one component from each layer of the set of M layers.

In some implementations, said plurality of identifier nucleic acid molecules is obtained by programmably synthesizing multiple oligonucleotides with de novo synthesis.

In some implementations, said plurality of identifier nucleic acid molecules is obtained by synthesizing degenerate oligonucleotide sequences.

In some implementations, the method includes incorporating common primer binding sites to each identifier molecule of the plurality of identifier nucleic acid molecules. In some implementations, the method includes amplifying the plurality of identifier nucleic acid molecules with polymerase chain reaction (PCR) using PCR primers configured to bind to said common primer sites.

In some implementations, the method includes adding a spacer sequence to each identifier nucleic acid molecule of the plurality of identifier nucleic acid molecules. In some implementations, the spacer sequence is added by one of ligation or overlap extension PCR. In some implementations, the spacer sequence is inserted into a target insertion site within the identifier nucleic acid sequence.

In some implementations, the spacer sequence is configured to increase a translocation time of each identifier nucleic acid molecule of the plurality of identifier nucleic acid molecules during sequencing in the nanopore system.

In some implementations, the nanopore system includes a source chamber, a membrane, a nanopore, and the destination chamber. In some implementations, accepting the identifier nucleic acid molecule includes translocating the identifier nucleic acid molecule from the source chamber to the destination chamber through the nanopore in the membrane. In some implementations, sequencing the identifier nucleic acid molecule includes detecting an impedance signal and matching the impedance signal to one of multiple impedance signatures. In some implementations, the methods includes binding an agent to each identifier nucleic acid molecule of at least a subset of the plurality of identifier nucleic acid molecules to provide a distinct impedance signal. In some implementations, the binding includes binding the agent to each identifier nucleic acid molecule of the plurality of identifier nucleic acid molecules.

In some implementations, the identifier nucleic acid molecule is accepted or rejected into the destination chamber based on at least one impedance signature to which the identifier nucleic acid molecule matches.

In some implementations, rejecting the identifier nucleic acid molecules includes reversing a polarity of an electric field across the nanopore.

In some implementations, the method includes sequencing multiple identifier nucleic acid molecules in the nanopore system until the destination chamber includes a plurality of identifier nucleic acid molecules that is sufficient for representing the digital information with error correction.

In some implementations, mapping includes using forward error correction.

In some implementations, the method includes correcting for any errors that occur during the sequencing step or the accepting or rejecting step by using backward error correction.

In some implementations, the destination chamber is a first destination chamber, and the target set is a first target set. In some implementations, the method further includes: accepting or rejecting the identifier nucleic acid molecule into a second destination chamber based on whether or not the identifier nucleic acid molecule corresponds to an identifier nucleic acid sequence of a second target set. In some implementations, the nanopore system includes a source chamber, a first membrane, a first nanopore in the first membrane, a second membrane, and a second nanopore in the second membrane. In some implementations, the first membrane separates the source chamber and the first destination chamber, and the second membrane separates the source chamber and the second destination chamber.

In some implementations, if said bit has a bit-value of 1, said bit is represented by a presence of the at least one corresponding identifier nucleic acid sequence in the first target set, and if the bit has a bit-value of 0, said bit is represented by a presence of the at least one corresponding identifier nucleic acid sequence in the second target set.

In some implementations, the method includes designating a probe set of component nucleic acid sequences. In some implementations, the methods includes sequencing a probed identifier nucleic acid molecule from the first destination chamber or the second destination chamber with the nanopore system. In some implementations, the method includes accepting or rejecting the probed identifier nucleic acid molecule into a retrieval chamber based on whether or not the probed identifier nucleic acid molecule corresponds to an identifier nucleic acid sequence containing a component nucleic acid sequence of the probe set.

In some implementations, accepting or rejecting the identifier nucleic acid molecule includes accepting the identifier nucleic acid molecule into the destination chamber if the identifier nucleic acid molecule has an identifier nucleic acid sequence of the target set. In some implementations, the method includes rejecting the identifier nucleic acid molecule from the destination chamber if the identifier nucleic acid molecule does not have an identifier nucleic acid sequence of the target set.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIGS. 1A and 1B schematically illustrate an example method of encoding digital data, referred to as “data at address”, using objects (components) or identifiers (e.g., nucleic acid molecules); FIG. 1A illustrates combining a rank object (or address object) with a byte-value object (or data object) to create an identifier; FIG. 1B illustrates an implementation of the data at address method wherein the rank objects and byte-value objects are themselves combinatorial concatenations of other objects (components);

FIGS. 2A and 2B schematically illustrate an example method of encoding digital information using objects (components) or identifiers (e.g., nucleic acid sequences); FIG. 2A illustrates encoding digital information using a rank object as an identifier; FIG. 2B illustrates an implementation of the encoding method wherein the address objects are themselves combinatorial concatenations of other objects (components);

FIGS. 3A and 3B illustrate an example method, referred to as the “product scheme”, for constructing identifiers (e.g., nucleic acid molecules) by combinatorially assembling distinct components (e.g., nucleic acid sequences); FIG. 3A illustrates the architecture of identifiers constructed using the product scheme; FIG. 3B illustrates an example of the combinatorial space of identifiers that may be constructed using the product scheme;

FIG. 4 schematically illustrates the use of overlap extension polymerase chain reaction to construct identifiers (e.g., nucleic acid molecules) from components (e.g., nucleic acid sequences);

FIG. 5 schematically illustrates the use of sticky end ligation to construct identifiers (e.g., nucleic acid molecules) from components (e.g., nucleic acid sequences);

FIG. 6 schematically illustrates the use of recombinase assembly to construct identifiers (e.g., nucleic acid molecules) from components (e.g., nucleic acid sequences);

FIG. 7 schematically illustrates the use of template directed ligation to construct identifiers (e.g., nucleic acid molecules) from components (e.g., nucleic acid sequences);

FIGS. 8A-8C schematically illustrate an overview of example methods for accessing portions of information stored in nucleic acid sequences by accessing a number of particular identifiers from a larger number of identifiers using probes; FIG. 8A shows example methods for using polymerase chain reaction, affinity tagged probes, and degradation targeting probes to access identifiers containing a specified component; FIG. 8B shows example methods for using polymerase chain reaction to perform ‘OR’ or ‘AND’ operations to access identifiers containing multiple specified components; FIG. 8C shows example methods for using affinity tags to perform ‘OR’ or ‘AND’ operations to access identifiers containing multiple specified components;

FIG. 9 illustrates an example of ordering identifiers in a combinatorial space. Identifiers in a combinatorial space with 4 layers and 2 components per layer are ordered by first sorting them according the components in layer 1, then the components in layer 2, then the components in layer 3, and then the components in layer 4. Line segments represent components. Further, grey segments represent the first component in each layer (j, 1), and black segments represent the second component in each layer (j, 2) where j is the layer. The composite line segments on the bottom represent full identifiers. For example purposes, the physical ordering of the components on each identifier here is the same as the logical ordering of the layers from which they are derived, but this need not generally be the case;

FIG. 10 illustrates an example of translating a 12 bit digital string into a pool of identifiers using a codebook that maps 6 bit words to codewords comprised of 8choose4 identifiers (4 identifiers chosen from a group of 8). In this particular example the 6-bit string 010010 maps to the codeword 01011100 and the 6-bit string 111011 maps to the codeword 11001001. Identifiers are ordered as explained in FIG. 9;

FIG. 11 shows identifiers in a combinatorial space with 3 layers and 2 components per layer, where identifiers of different lengths are included in the space. The identifiers are ordered by first sorting them according the number of layers that they contain. They are subsequently sub-sorted by the components in layer 1, then the components in layer 2, then the components in layer 3, and then the components in layer 4. Line segments represent components. Grey segments represent the first component in each layer, and black segments represent the second component in each layer. The composite line segments represent full identifiers. Identifier sequences of different lengths are included in the identifier space;

FIG. 12 illustrates how identifier sequences, represented by rectangles of different pattern fills, are logically ordered such that two identifier sequences map to the same position. The presence of an identifier molecules of either sequence in a pair would signify the presence of an identifier sequence at the given position;

FIG. 13 illustrates an identifier sequence comprised of components (represented by black and grey line segments) passing through a nanopore in a membrane. As the sequence passes through, an impedance signal is measured on a computer and used as a reference against a database of possible component signatures;

FIG. 14A illustrates how an identifier space is used to encode a codeword wherein a bit-value of 0 is represented by the exclusion of the corresponding identifier sequence from a pool, and a bit-value of 1 is represented by the inclusion of the corresponding sequencing in the pool. FIG. 14B illustrates how a nanopore device writes the codeword. The source chamber contains all identifier sequences from the identifier space. Only identifier sequences corresponding to bit-values of 1 in the codeword are allowed to pass into the destination chamber;

FIG. 15A illustrates how a spacer is inserted into an identifier sequence by first inserting the identifier into a vector, and then cutting the identifier sequence at a cut site. The identifier sequence is designed such that sequencing either half of the identifier sequence is sufficient for determining the entire identifier sequence. FIG. 15B shows an example of the mechanism by which a spacer permits suitable time for a nanopore system to determine whether to accept or reject an identifier molecule;

FIG. 16A illustrates how a nanopore device writes the codeword. The source chamber contains all identifier sequences from the identifier space. Only identifier sequences corresponding to bit-values of 1 in the codeword are allowed to pass into the bit-value “1” destination chamber. Only identifier sequences corresponding to a bit-value of 0 in the codeword are allowed to pass into the bit-value “0” destination chamber. FIG. 16B illustrates how A nanopore device writes the codeword. The bit-value “1” destination chamber contains all identifier sequences from the identifier space with a bit-value of 1. Once a subset of components are designated as probes to retrieve information, the library is sequenced and only identifiers with the probe sequence are moved into the new chamber (in this case the source chamber);

FIG. 17 shows a base module of a printer-finisher system, according to an illustrative implementation;

FIG. 18A shows a printer engine rack. FIG. 18B is a schematic of the positioning of print heads, according to an illustrative implementation;

FIG. 19 shows a diagram of dispensing solutions into compartments according to a trie of identifiers, according to an illustrative implementation;

FIG. 20 shows a pooling sub-system, according to an illustrative implementation;

FIG. 21 shows an archival information system, according to an illustrative implementation;

FIG. 22 shows a system diagram of an operating system organized by layer, according to an illustrative implementation;

FIG. 23 shows system diagram of a layered product constructor with three layers and a component library of eight components, according to an illustrative implementation;

FIG. 24 shows a flowchart for storing digital information into nucleic acid molecules with error protection, by appending hashes, according to an illustrative implementation;

FIG. 25 shows a flowchart for storing digital information into nucleic acid molecules with error protection, by separately storing hashes, according to an illustrative implementation;

FIG. 26 shows a flowchart for storing digital information into nucleic acid molecules, using a partition scheme, according to an illustrative implementation;

FIG. 27 shows a system diagram of a uniform weight code, according to an illustrative implementation;

FIG. 28 shows a system diagram of a decoding stack, according to an illustrative implementation;

FIG. 29 shows a flowchart for reading information stored in nucleic acid sequences, according to an illustrative implementation;

FIG. 30 shows a PCR amplification scheme, according to an illustrative implementation;

FIGS. 31A and 31B show identifier nucleic acid molecules with signal events, according to an illustrative implementation;

FIG. 32 shows a system diagram of archival operations, according to an illustrative implementation;

FIG. 33 shows a system diagram of writing data, according to an illustrative implementation; and

FIG. 34 shows a flowchart for storing blocks of data in containers, according to an illustrative implementation.

### DETAILED DESCRIPTION

To provide an overall understanding of the systems, method, and devices described herein, certain illustrative embodiments will be described. Although the embodiments and features described herein are specifically described for use in connection with nucleic acid-based data storage, it will be understood that all the components and other features outlined below may be combined with one another in any suitable manner and may be adapted and applied to other types of data storage and nucleic acid technology.

There is a need for nucleic acid digital data storage encoding and retrieving methods that are less costly and easier to commercially implement than existing methods. The systems, devices, and methods described herein provide scalable methods for writing data to and reading data from nucleic acid molecules. The present disclosure covers four primary areas of interest: (1) accurately and quickly reading information stored in nucleic acid molecules, (2) partitioning data to efficiently encode data in nucleic acid molecules, (3) error protection and correction when encoding data in nucleic acid molecules, and (4) data structures to provide efficient access to information stored in nucleic acid molecules.

Identifiers are nucleic acid sequences that encode digital information. For example, an identifier may represent a symbol in a string of symbols. Identifiers may include component nucleic acids, which can be configured to bind a probe. We may refer to component nucleic acid sequences, configured as such, as “addressable components”. All components as described herein may be addressable components. The term “probe,” as used herein generally refers to an agent that binds a target sequence on an identifier nucleic acid molecule. The target sequence can be a portion of a component. The probe can include a sequence that matches or is the complement of its target sequence. The probe can further be used to isolate all identifier nucleic acid molecules including said target sequence. For example, the probe can be a primer in a PCR reaction that enriches all identifier nucleic acid molecules including a target sequence. Or the probe can contain an affinity tagged oligonucleotide molecule that can be used to select all identifier nucleic acid molecules with a sequence that corresponds to said oligonucleotide. For example, a probe may be a biotinylated oligo that binds its complementary target and is subsequently captured by a streptavidin bead or column. A probe can be used for negative selection as well. For example, an affinity tagged probe can be used to remove all identifiers containing a particular target sequence. Or, alternatively, a probe can include an active nuclease, such as Cas9, that cleaves or digests all identifiers containing a particular target sequence.

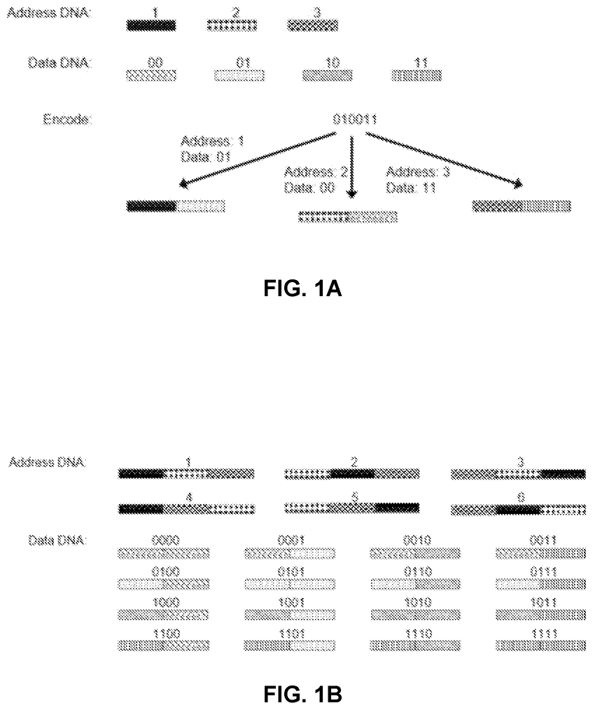

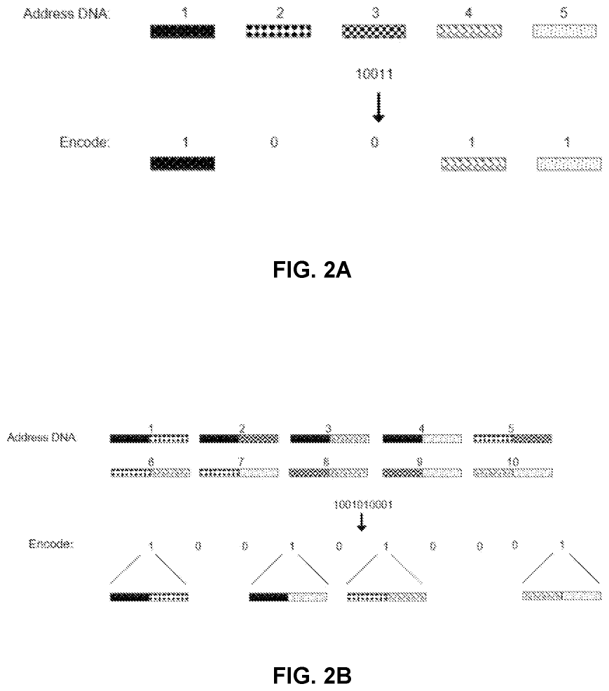

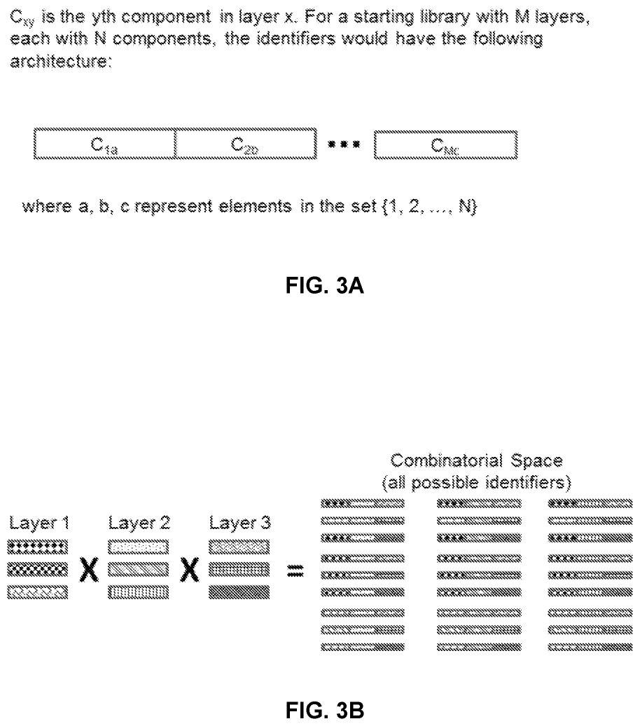

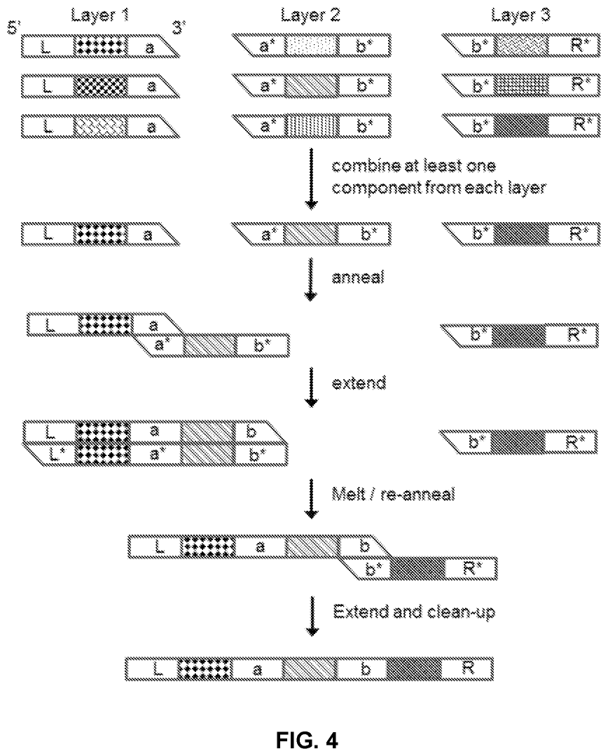

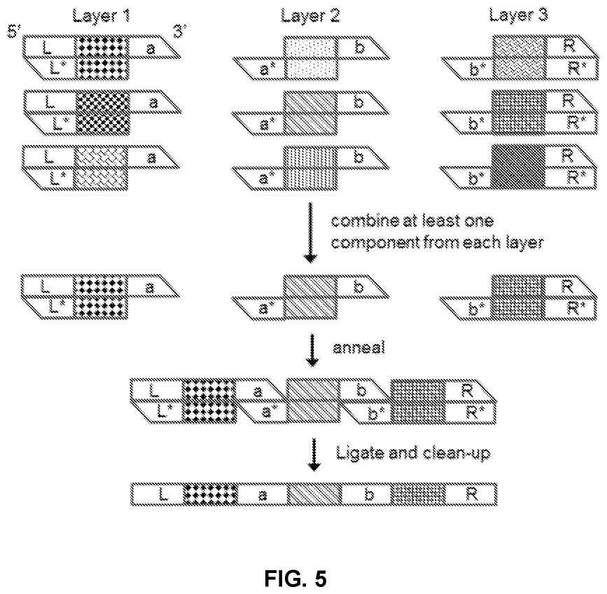

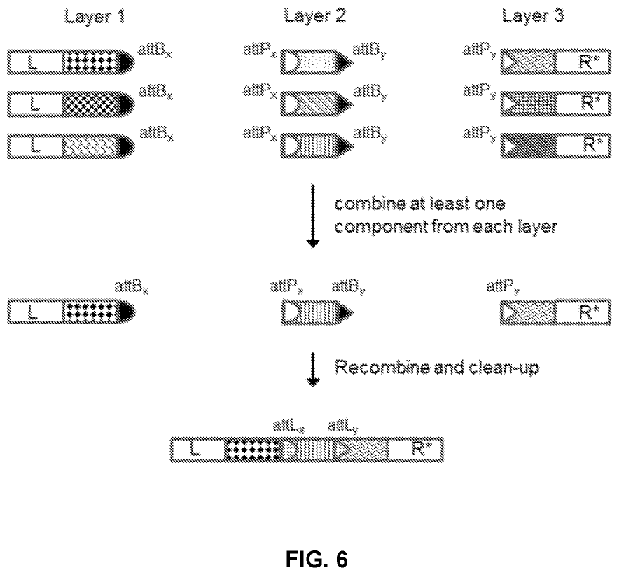

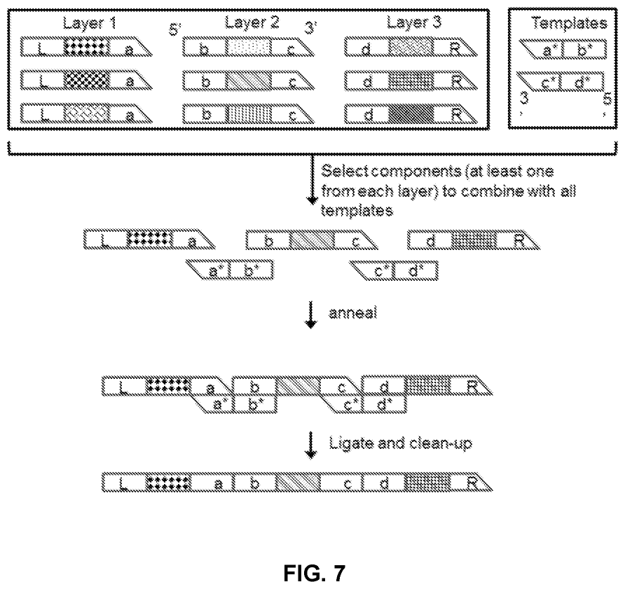

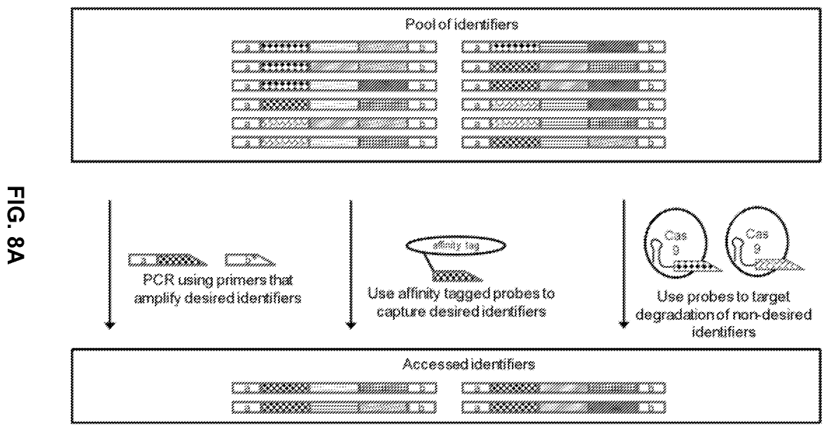

FIGS. 1 and 2 illustrate examples of how identifiers including components can encode digital information. FIG. 3 illustrates an example scheme, termed the “product scheme” for constructing identifiers from components, where components are divided into layers and identifiers are constructed from assembling one component from each layer. The combinatorial space, or identifier space, is the set of all possible identifiers that can be formed from a particular scheme. A subset of this space is constructed to encode digital information. This subset may be referred to as an identifier library or pool. FIGS. 4-7 illustrate example chemistries for executing the product scheme. FIG. 8 illustrates examples of using probes to access specified subset of identifiers from an identifier library.

In some embodiments, the identifiers may be comprised entirely of addressable components. The addressable components may be assembled to form an identifier or they may be introduced into an identifier sequence through subtractive or substitution approaches. Alternatively, they can be incorporated into a nucleic acid identifier by de novo synthesis. Different writing methods vary in speed and cost. They can also vary in the number of possible components that can be incorporated into an identifier. Techniques for constructing identifiers, mapping data to identifiers, accessing a specified set of identifiers using probes, and reading identifiers are described in U.S. Pat. No. 10,650,312 entitled “NUCLEIC ACID-BASED DATA STORAGE”, filed Dec. 21, 2017 (describing encoding digital information in DNA); U.S. application Ser. No. 16/461,774 entitled “SYSTEMS FOR NUCLEIC ACID-BASED DATA STORAGE”, filed May 16, 2019 and published as U.S. Publication No. 2019/0362814 (describing encoding schemes for DNA-based data storage); U.S. application Ser. No. 16/414,758 entitled “COMPOSITIONS AND METHODS FOR NUCLEIC ACID-BASED DATA STORAGE”, filed May 16, 2019; U.S. application Ser. No. 16/532,077 entitled “SYSTEMS AND METHODS FOR STORING AND READING NUCLEIC ACID-BASED DATA WITH ERROR PROTECTION”, filed Aug. 5, 2019 (describing data structures and error protection and correction for DNA encoding); and U.S. application Ser. No. 16/872,129 entitled “DATA STRUCTURES AND OPERATIONS FOR SEARCHING, COMPUTING, AND INDEXING IN DNA-BASED DATA STORAGE”, filed May 11, 2020 (describing data structures and operations for access, rank, and search), the contents of each of which are hereby incorporated by reference in their entireties.

Encoding Bits into Identifiers

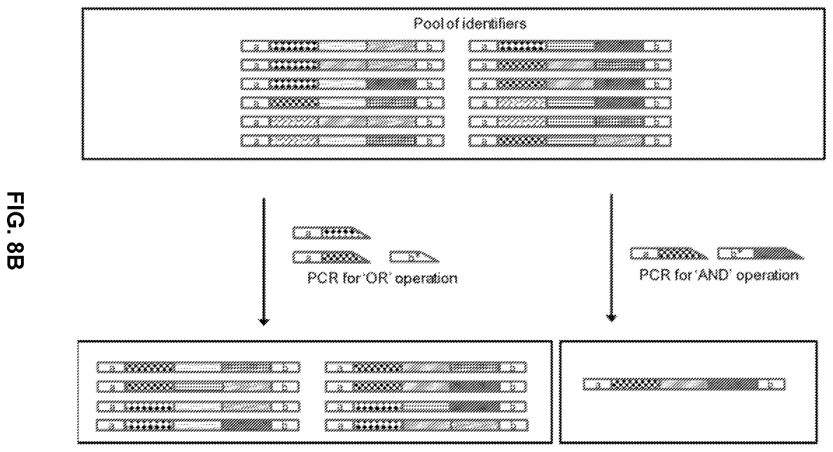

Each identifier in a combinatorial space can include a fixed number of N components where each component comes from a distinct layer in a set of N layers, and is one of a number of a set of possible components in said layer. Each component can be specified by a coordinate (j, Xⱼ) where j is the label of the layer and Xⱼ is the label of the component within the layer. For said scheme with N layers, j is an element of the set {1, 2, . . . , N} and Xⱼ is an element of the set {1, 2, . . . , Mⱼ) where Mⱼ is the number of components in layer j. We can define a logical order to the layers. We can also define a logical order to each component within each layer. We can use this labeling to define a logical ordering to all possible identifiers in the combinatorial space. For example, we can first sort the identifiers according to the order of the components in layer 1, and then subsequently according to the order of the components in layer 2, and so on, as shown in FIG. 9.

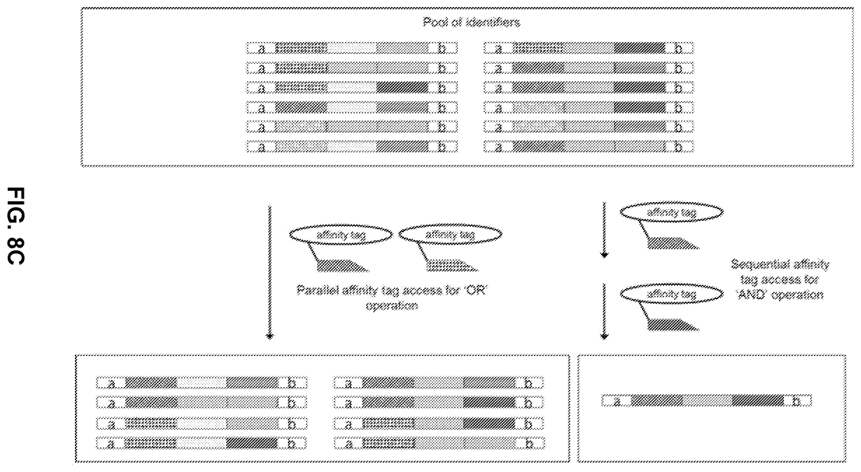

The logical ordering of the identifiers can be further used to allocate and order digital information. Digital information can be encoded in nucleic acids that include each identifier, or it can be encoded in the presence or absence of the identifiers themselves. For example, we can create a codebook that encodes 4 bits of information in every contiguous grouping of 4 identifiers. In this example, the codebook could map each possible string of 4 bits to a unique combination of 4 identifiers (since there are 16 possible combinations of 4 identifiers, it is possible to store up to log₂(16)=4 bits of data). As another example, we can create a codebook that encodes 6 bits of data in every contiguous group of 8 identifiers. In this example, the codebook could map each possible string of 6 bits to a unique subsets of 4 out of the 8 identifiers (since there are 8 choose 4=70 such subsets, it is possible to store up to floor(log₂(70))=6 bits of data). We may refer to these identifier combinations as codewords and we may refer to the data that they encode as words. Adjacent words within data may be stored in adjacent codewords among the logically ordered identifiers. FIG. 10 shows an example of translating a 12 bit digital string into a pool of identifiers using a codebook that maps 6 bit words to codewords comprised of 8choose4 identifiers (4 identifiers chosen from a group of 8). Codewords can be represented symbolically as bit strings where every bit position corresponds to an ordered identifier, where the bit-value of ‘0’ represents the absence of the corresponding identifier in the codeword and the bit-value of ‘1’ represents the presence of the corresponding identifier in the codeword.

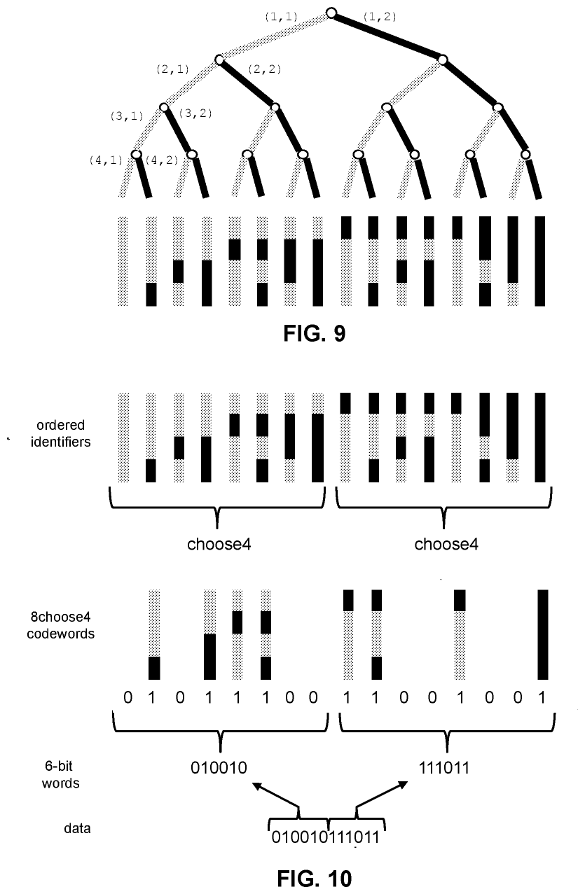

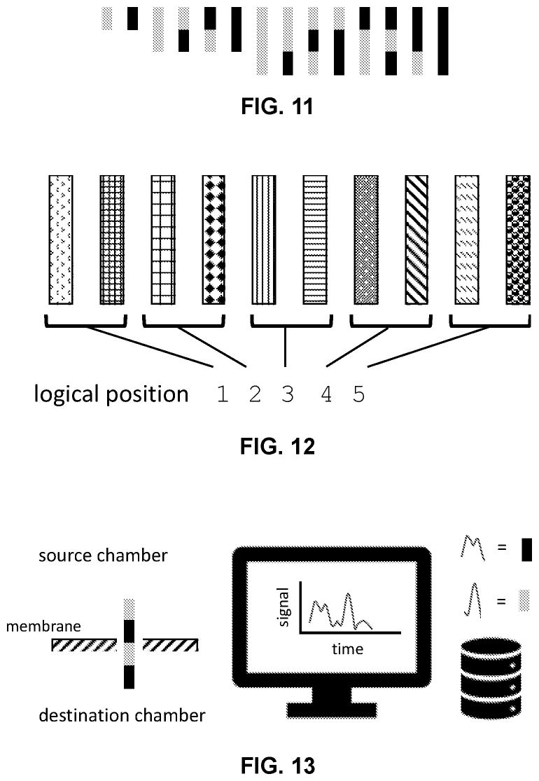

We can generalize this encoding scheme to include identifier sequences of arbitrary lengths and sequence composition. For example, as illustrated in FIG. 11, we can allow for identifier sequences containing different numbers of components and lexicographically order the identifier sequences such that identifiers of a given sequence logically precede all identifier sequences of which they are a prefix. This is analogous to how words are ordered in the English dictionary. Further, we can define a component as a base. For example, we can use the 4 natural bases A, G, C, and T as four separate components within a layer given by the position of the base. Using such a scheme and assigning a possible layer for every possible base position, we can include all possible DNA sequences as identifiers. For example, the ordering of all possible DNA sequences would be A, C, G, T, AA, AC, AG, AT, CA, CC, CG, CT, GA, GC, GG, GT, TA, TC, TG, TT, AAA, AAC, AAG, AAT, ACA, ACC, ACG, ACT, and so on. In another embodiment, we may preserve this identifier order, but choose to constrain the identifier space to a fixed subset of these sequences. For example, we can constrain the identifier space to all sequences that have between 50 and 100 bases. Or we can constrain our identifiers to all sequences that have particular bases in particular positions. More generally, we can choose any arbitrary set of sequences as our identifier space and define their ordering using any arbitrary dictionary. In some embodiments multiple identifier sequences may map to same logically ordered position. An example is shown in FIG. 12. We call this a “degenerate encoding scheme” as multiple sequences are treated as if they were the same.

### Writing-by-Sequencing

Using the encoding schemes described above, one may write digital information into nucleic acids by creating physical pools of identifier molecules corresponding to subsets of identifier sequences from a larger identifier space. Systems for creating identifier pools can be constructive—relying on constructing each identifier sequence within the subset and excluding the construction of each identifier sequence not in the subset. Other approaches can be subtractive—relying on creating all identifier sequences of an identifier space and then selectively removing all identifier sequences that do not belong in the subset. Here we describe a subtractive approach wherein nanopore sequencing is used to perform the sequence selection.

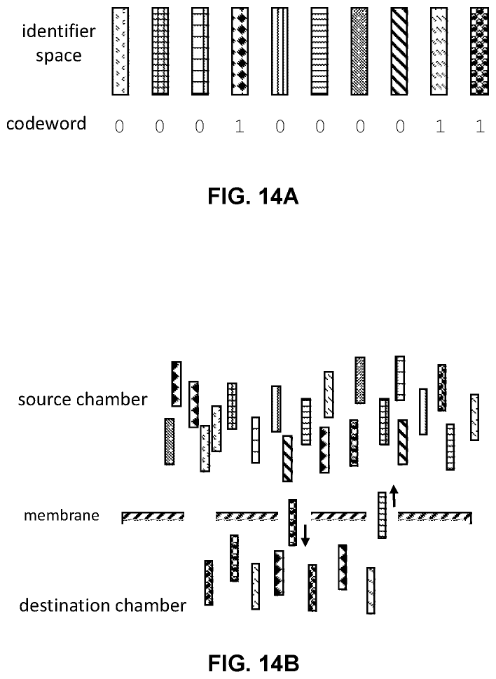

Nanopore sequencing may include a system wherein an electric field is applied to an electrolytic solution and at least one nanopore channel separating two chambers, a source chamber and a destination chamber. In some implementations, the nanopore channel is formed within a solid state membrane. In some implementations, the nanopore channel is formed from alpha-hemolysin (αHL) or Mycobacterium smegmatis porin A (MspA). During nanopore sequencing, an identifier molecule in the source chamber is translocated through a nanopore channel, while impedance across the channel is measured. Each identifier sequence has a corresponding unique impedance signature, thus allowing it to be identified using the impedance values from the nanopore as it translocates across. An example of nanopore sequencing of an identifier is shown in FIG. 13. If the identifier sequence is comprised of components, then each component in the identifier may have a corresponding unique impedance signature, thus allowing the component identities to be determined by comparing measured impedance values to the unique impedance signature.

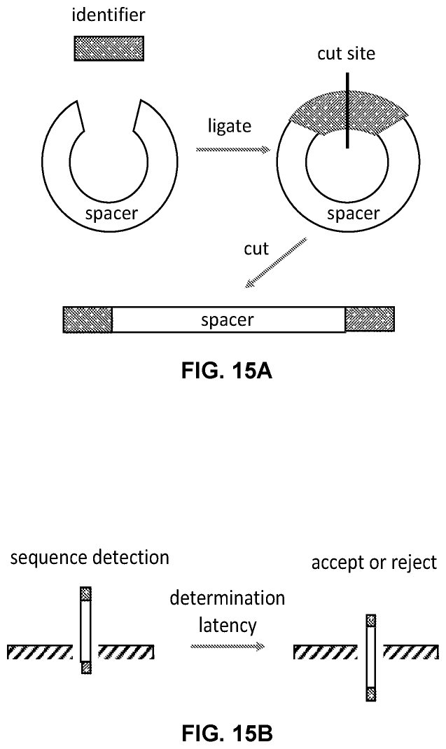

Such impedance signatures can be stored in a database and matched to reference impedance values in real-time as the identifier molecule translocates across the nanopore. Based on this matching process, a determination can be made to either accept the molecule to continue translocating into the destination chamber, or to reject the molecule and reverse the translocation such that the molecule remains in the source chamber. The minION™, GridION™, and PromethION™ sequencers from Oxford Nanopore Technologies™ are examples of sequencers that can be used for this process; however, other nanopore sequencing technologies, including nanopore technologies that are capable of real-time selective sequencing by reversing the polarity across a nanopore, may be used. This selective technology, sometimes referred to as “Read Until”, has been demonstrated for genomic applications (see Edwards, H. S., et al. “Real-Time Sequencing with RUBRIC: Read Until with Basecall and Reference-Informed Criteria.” Sci Rep. 2019; 9: 11475, which is hereby incorporated by reference in its entirety), but not applied towards writing digital information into DNA as described here. We can load identifier molecules corresponding to a full identifier space in the source chamber and then use real time selective sequencing to ensure that molecules corresponding to identifier sequences in a targeted subset pass through a nanopore into the destination chamber while identifier sequences that do not correspond to identifier sequences in the targeted subset are rejected from entering the destination chamber. After sufficient time, the resulting destination chamber will correspond to a pool of identifiers that stores digital information as per the encoding schemes described above. An example of the writing process is illustrated in FIG. 14. The pool of identifiers may then be amplified with PCR and used for downstream processes, such as computing, random access, or reading.

In some implementations, the applied electric field across the nanopore generates a differential potential greater than or equal to 100 mV. This high differential potential enables the identifier to be passed through the nanopore channels at a relatively high rate, for example, compared to rates using potentials of less than 100 mV. For example, translocation of the identifier may occur at a rate great than 1,000 bases per second. In particular, the translocation rate may be 1,000,000 bases per second. Additionally, multiple nanopores may be used in parallel to increase throughput of writing.

In some implementations, an agent is bound to the identifier before translocating. For example, the agent may be a chemical moiety, a protein, an enzyme, a base analogue, a conjugated nucleic acid, a nucleic acid with a hairpin, or a methyl group. In some implementations, if the agent is a chemical moiety, an enzyme, such as methyltransferase, binds the chemical moiety to the at least one identifier nucleic acid molecule. In some implementations, if the agent is a base analogue and the agent is bound using an enzyme, such as a polymerase, the enzyme binds the base analogue to the at least one identifier nucleic acid molecule during replication.

The agent is associated with an agent signature that can be used to help determine sequences in the identifier during reading. Binding the agent to the at least one identifier nucleic acid molecule occurs at a known location on a component of the identifier, such that the agent signature at the known location results in a known shift in impedance value during translocation. The presence of the agent may thus create an exaggerated “profile” for the identifier, thereby increasing the signal-to-noise ratio during reading. This may allow the translocation speed to be increased while maintaining accuracy during reading and writing.

Current nanopore sequencer flow cells, for example, from Oxford Nanopore Technologies™, are capable of sequencing up to 200 Gigabases of DNA or more in a couple days. If each identifier is around 200 bases, then a sequencing run on such a flow cell would yield around 1 billion total identifier sequences queried for membership in the final identifier pool. Such a method could be used to encode up to one gigabit of information. But because the sequencing is a random process and some sequences will be sequenced multiple times while others are not sequenced at all, this will result in codeword erasures or errors in the encoded information. The incidence of such erasures or errors can be decreased by increasing the sequencing coverage, for example, by using the approximately one billion sequence reads to encode 100 megabits of information instead of one gigabit of information. The remaining chance of errors and erasures can be corrected for with error correction. Such error correction can be forward error correction, such as Reed-Solomon coding, which encodes information with error tolerance up to a certain threshold, or with backward error correction methods, which track errors that occurs during the writing process and fix them afterwards.

The ability to do backward-error correction is unique to this writing method since the reading and writing occur simultaneously. This enables the system to keep track of every identifier sequence that was supposed to be in the final identifier pool but that was never included or conversely every sequence that was not supposed to be in the final identifier pool, but that was accidentally included. Such metadata can be stored along with the encoded information, either by conventional means, or in a separate identifier pool. It is likely that such metadata will be much smaller in size than the total data.

More generally, the ability to confirm the written identifier sequences by reading them as they are written is unique to this method. Not only does this enable backward error correction, but it enables more rigid quality control compared to other methods where the writing and reading occur in two different steps. For example, a failed or low quality run may be determined part-way through the writing process, in which case the system can terminate the process, report the termination, reset the system and reagents, and start again. This is more akin to traditional information writing processes where the reading occurs tangential to the writing and may be used for proof-reading. This also creates a technology development ecosystem whereby reading and writing processes can be improved together, since they utilize the same system. As the speed, throughput, accuracy, and convenience of nanopore sequencers improve for reading information from DNA, so too would they improve at writing information into DNA. Another benefit of this approach is the footprint of the system. Nanopore sequencers, for example, the MinION™ from Oxford Nanopore Technologies™, can be handheld and can also run on smartphones.

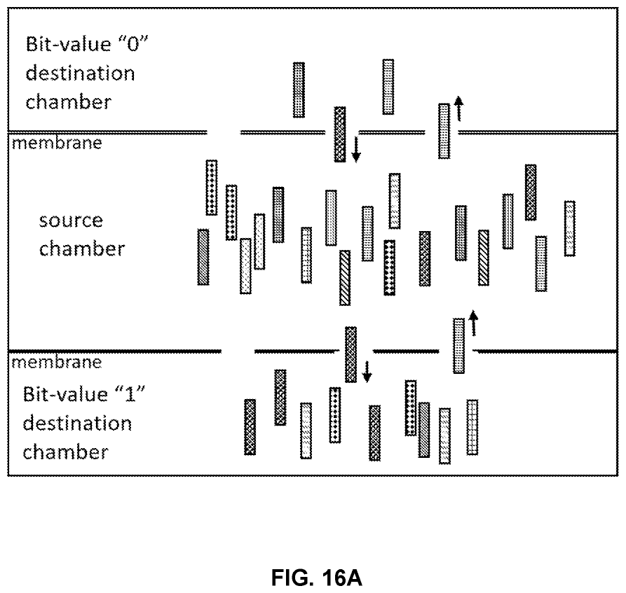

The full space of identifier sequences that go into the source chamber may be created in bulk and re-used for multiple instances of writing. Multiple strategies may be used for creating these identifier sequences quickly, cheaply, and reliably. In one embodiment, the identifier sequences may be created through assembly from component parts using the product scheme. For example, all component molecules of all layers may be mixed together in a large, multiplexed assembly reaction, as illustrated in FIG. 15. For example, an identifier space of 1 billion sequences can be constructed by mixing together 9 layers of 10 components each. As long as all components assemble with similar efficiency, and all components are loaded into the large reaction at similar stoichiometries, then we would expect to obtain a relatively uniform distribution of all identifier sequences in the resulting product pool. Care should be taken to account for the diversity of the identifier space when determining how many component molecules to load. For example, if the assembly efficiency of a full identifier sequence is 1% and there are a total of 1 billion identifier sequences in the space, then at least 100 billion molecules of each layer of components should be added to the large reaction in order to create at least one molecule of each identifier sequence in the identifier space.

In another embodiment, all possible identifier sequences of a space may be programmably synthesized de novo from the constituent bases. Such programmable synthesis may be done in high-throughput by array synthesis, where multiple oligos are synthesized in parallel on a chip, and then removed from the chip and pooled together. Such pooled oligo libraries are available from multiple suppliers and typically range from 10K-1M oligos of lengths up to 200 bases. Such methods could yield an identifier space, for example, of up to 1M identifier sequences, each at 150 bases. Extra processing steps may yield more sequences. For example, the 150-base identifier sequences may be designed such that they can be cleaved using endonucleases into smaller segments of around 50 bases each, Then the identifier space may be transformed into 3M identifier sequences of 50 bases. This programmable, de-novo synthesis method, though comparatively low throughput compared to the aforementioned component assembly method, enables the design of identifier sequences at base resolution. The sequences may be designed to work particularly well for downstream data applications after the information is written into DNA. For example, any form of computation that requires melting full identifier sequences and then hybridizing them back together may benefit from the ability to design each identifier sequence to be maximally orthogonal to every other identifier sequence in terms of thermodynamic interactions.

In another embodiment, all possible identifiers sequences of a space may be created through degenerate oligo synthesis. In degenerate oligo synthesis, all possible combinations of bases at particular positions of an oligo are generated by inputting equal mixtures of A, G, C, and T bases during the oligo synthesis process. This synthesis approach obviates the need to synthesize each oligo separately, and it is capable of synthesizing a much larger diversity of identifier sequences compared to the programmable, de novo synthesis approach. For example, degenerate bases at 20 different positions of an oligo can be specified, resulting in the multiplexed formation of 420 (greater than a trillion) possible identifier sequences in a single pool. This method, while advantageous over programmable synthesis in its ability to create many DNA sequences, is limited in sequence design capacity. For example, all identifier DNA sequences created from this process are always just one base mutation away from multiple other identifier sequences. This proximity between identifiers in edit distance may lead to multiple errors during the writing, storing, and reading process. This risk may be counteracted by a degenerate encoding scheme that treat similar identifier sequences as one (see FIG. 12).

Once the identifier sequences of an identifier space have been constructed, they may be designed with common primer binding regions on each end, or with the ability to append common primer binding sequences to each end. These common regions may be used to amplify the full library of identifiers sequences indefinitely. In other words, once a full identifier space is constructed, it may be replicated to produce multiple copies that may then be used in multiple instances of writing information.

The method for writing-by-sequencing described herein depends on the system being able to determine whether or not an identifier molecule belongs to a targeted subset of identifier sequence while the molecule is translocating across a nanopore. If a determination is made after the molecule has fully translocated into the destination chamber then it cannot be selectively reversed into the source chamber. In order to prevent this and increase the likelihood that the accept-or-reject decision for an identifier molecule is made while the molecule is still translocating, we can artificially insert a spacer sequence into the identifier so that it translocates for a longer period of time after the relevant portion of the identifier molecule has passed through the nanopore. In a nanopore system where a DNA molecule can only be sequenced from one end, then the spacer sequence need only be appended to the opposite end of the identifier. This may be accomplished through various assembly methods, such as ligation or overlap extension PCR. In a nanopore sequence wherein a DNA molecule may be sequenced from either end with equal probability, then the spacer sequence must be inserted into an identifier sequence, and the insertion point in the identifier sequence must be such that the resulting sequence information on one-end the spacer necessarily implies the sequence information on the other end, such that reading either end of the identifier sequence would suffice in reconstructing the entire identifier sequence. This may be accomplished using insertion methods with integrases that are capable of inserting spacer sequences into linear identifier sequences. In another embodiment, the linear identifier sequence may be inserted into a circular vector including a spacer sequence, and then cleaved, for example with an endonuclease, at a target site within the identifier sequence. The resulting molecule will have a unique identifier sequence on each end, either of which may pass through a nanopore first and be used to make an accept-or-reject determination as the rest of the molecule continues to transport through. An illustration of this method is shown in FIG. 15. The length of the spacer sequence can be programmed for compatibility with the sequence determination latency and the speed of the translocation. For example, if the determination process requires at most 3 s, and the translocation speed is 300 bases per second, then the spacer sequence should be at least 900 bases in length. After the writing process is complete, the spacer sequences may be removed from the molecules, for example, by endonuclease digestion or by PCR-ing the relevant identifier sequences.

To make the writing process even more turn-key, starting identifier libraries (with spacers included if necessary) may be pre-prepped for sequencing and pre-loaded into source chambers. With this preparation, the writing-by-sequencing process can occur on-demand and in the field without any preliminary reagent handling. A user could simply send data to the system and execute a write instance.

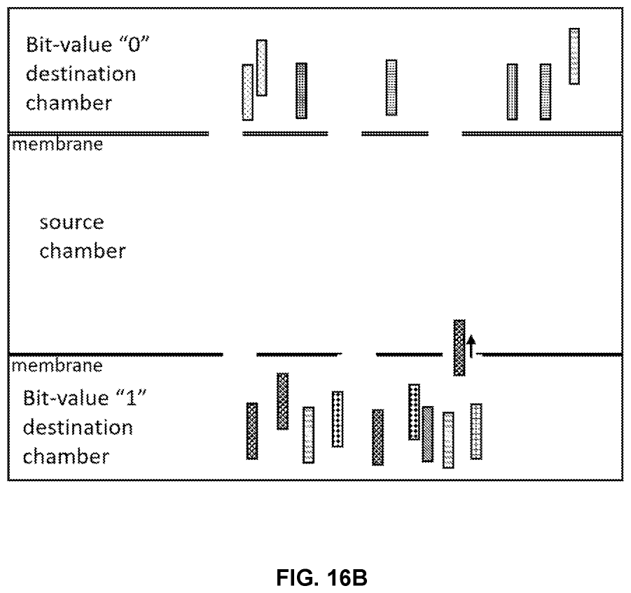

Additional chambers can be used to improve the efficiency of the initial writing-sequencing step by reducing the complexity of the starting identifier library. One of the more time consuming steps in nanopore sequencing is the time required to associate the nucleic acid with the nanopore. If a particular nucleic acid is sequenced and rejected from the nanopore, for example, identifiers with a bit-value of “0”, then the starting pool of identifiers will continue to be enriched for identifiers with a “0” bit-value. This enrichment will make it less likely to find identifiers with a bit-value of “1”. Using multiple chambers separated by nanopores, it is possible to mitigate this enrichment. One example is shown in FIG. 16A. Identifiers corresponding to a bit-value of “0” are sorted into a first destination chamber while identifiers corresponding to a bit-value of “1” are sorted into a second destination chamber. Each destination chamber may be controlled by a distinct nanopore or set of nanopores. Sorting into each destination chamber may be performed in parallel, such that the source chamber maintains an approximately constant enrichment of identifiers corresponding to each bit-value of “0” and “1”. Similarly, this multi-chamber approach can be used to select a subset of identifiers from an encoded library (FIG. 16B) in order to sequence and decode a specific piece of information. Finally, it is possible to rebuild the original library by combining all separated identifiers—effectively “deleting” the encoded information via modulation of the nanopores.

While the reading methods described herein may be used to read any nucleic acid sequence, the reading methods of the present disclosure are particularly advantageous when reading information stored in nucleic acid sequences that were written into said sequences using an encoding method that writes data or information in identifier nucleic acid molecules (also referred to herein as simply “identifiers” or “identifier molecules”). The nucleic acid sequence of each identifier molecule corresponds to a particular symbol value (e.g., bit or series of bits), that symbol's position (e.g., rank or address), or both, in a string of symbols (e.g., a bit stream). For example, the presence or absence of an identifier molecule could signal a bit value of 1 or 0, respectively (or vice versa). The identifier nucleic acid molecules include combinatorial arrangements of component nucleic acid molecules (also referred to herein as simply “components” or “component molecules”). In some implementations, the nucleic acid sequences of the components are separated into unique sets, also referred to as layers. Identifier molecules are assembled by ligating together (or otherwise assembling) multiple component molecules, one component molecule selected from each layer. The set of possible identifier sequences corresponds to the various possible combinatorial combinations of the component sequences. For example, for C component sequences separated into M layers, with cᵢ representing the number of component sequences in each i^{th} layer, the number of possible identifier sequences that can be formed can be represented by c₁×c₂× . . . ×c_{M}. As an example, an encoding scheme of 12 layers, each containing 10 component sequences can result in 10¹² different unique identifier sequences. If each identifier sequence corresponds to a bit in a bit stream, this encoding scheme can represent 1 Tb of data. Examples of various methods of writing digital information into nucleic acid molecules are in U.S. application Ser. No. 15/850,112 entitled “NUCLEIC ACID-BASED DATA STORAGE”, filed Dec. 21, 2017 and published as U.S. Patent Publication No.: 2018/0137418; U.S. application Ser. No. 16/461,774 entitled “SYSTEMS FOR NUCLEIC ACID-BASED DATA STORAGE”, filed May 16, 2019; and U.S. application Ser. No. 16/414,758 entitled “COMPOSITIONS AND METHODS FOR NUCLEIC ACID-BASED DATA STORAGE”, filed May 16, 2019, each of which is hereby incorporated by reference.

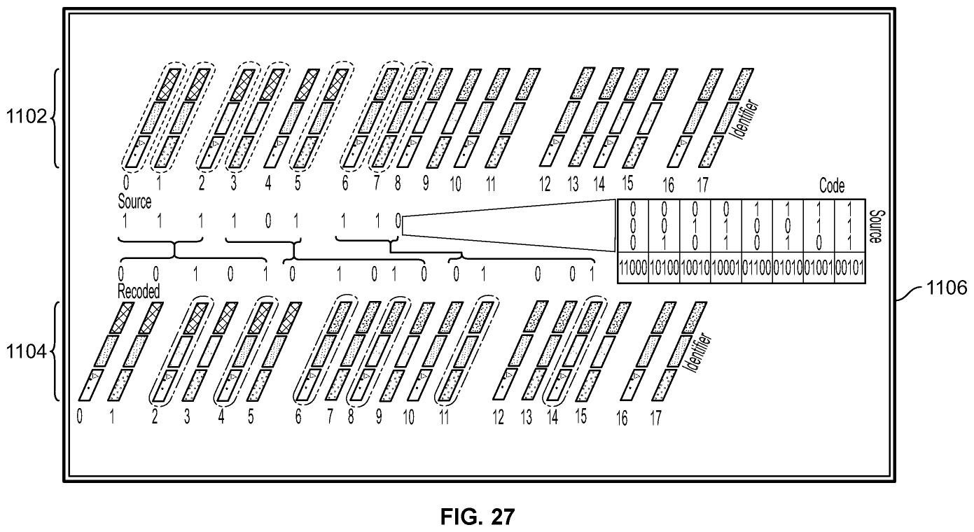

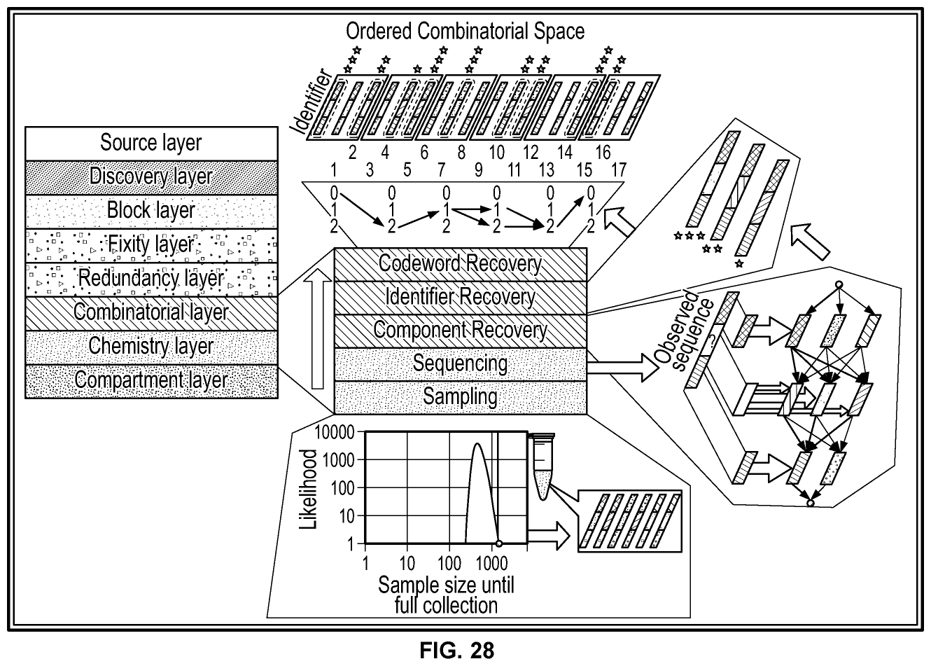

Sequencing or reading nucleic acid molecules is often error-prone due to difficulty distinguishing between nucleotides (for example, because of a poor signal-to-noise ratio). Because symbols encoded using the above-described methods are represented as identifier nucleic acid molecules that are formed from a set of component molecules that are known a priori, reading a sequence of a given molecule to determine the information encoded therein does not require an accurate reading of each and every single base in that sequence. Instead, the reading method disclosed herein can tolerate a relatively high error rate, and still correctly decode the digital information from the nucleic acid molecules. To do that, portions of sequences that have been read can be matched to the known set of component sequences by using an approximate string matching technique to determine which symbol in the string of symbols is most likely to be represented by the observed (or read) identifier molecule. In some implementations, the component sequences are designed so that each component sequence is separated from each other component sequence by at least a minimum number of base differences (for example, a minimum Hamming distance or Levenshtein distance). Requiring the component sequences to be distinct from one another in this manner reduces the chance that one sequence of a component molecule being sequenced will be mistaken for another component sequence when matching sequences. The reading system of the present disclosure is therefore robust (e.g., less sensitive to base errors), and identifier molecules can be read at a faster rate and with fewer errors than in traditional sequencing, as is explained in further detail below, with reference to FIG. 28 and FIG. 29.

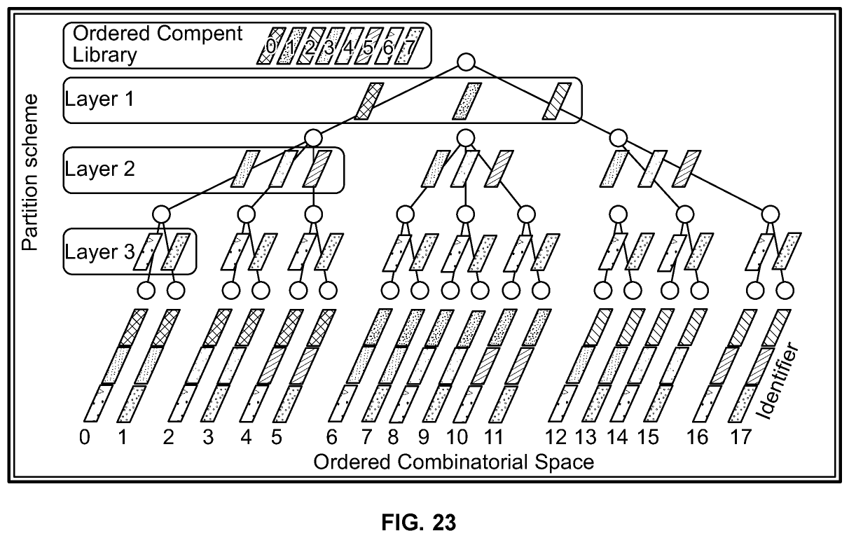

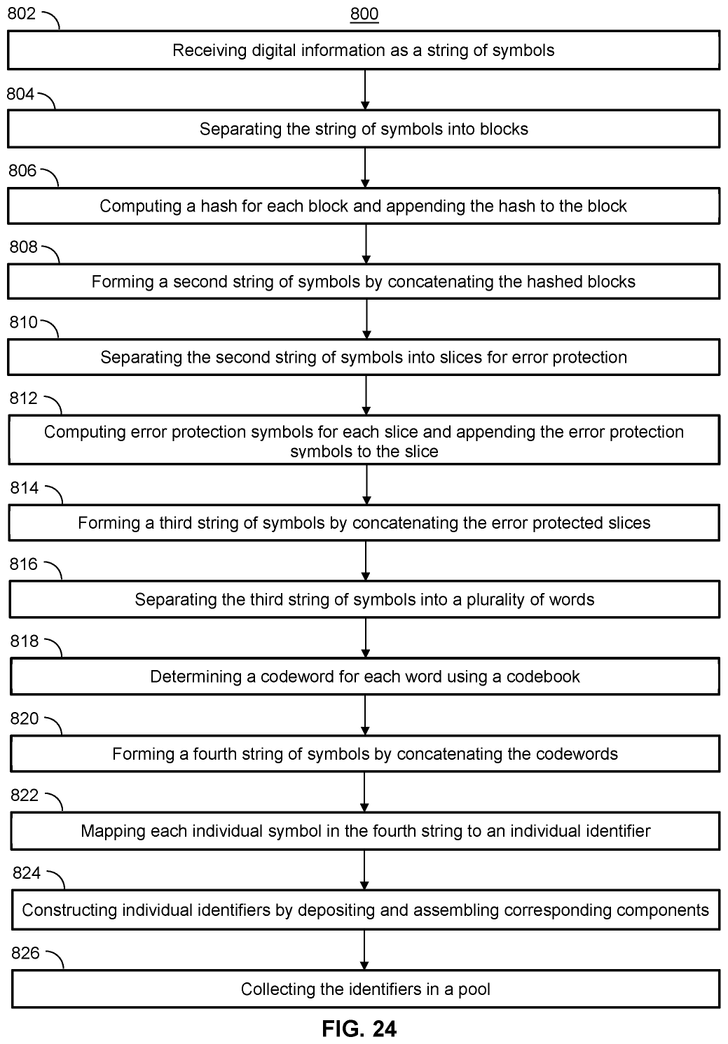

One way to improve tolerance to errors when reading data stored in nucleic acid molecules is to include error protection symbols and error correction schemes when encoding the data. To accomplish this, the source data (e.g., the string of symbols) is split into blocks, a hash is calculated for each block, and the hashes are appended to the source data at the end of each block to obtain a modified string of symbols, which is written into DNA. When the portion of the modified string of symbols corresponding to one of those hashes is read out from the DNA, it is compared to a hash computed on the read out symbols of the corresponding block. A mismatch between the read out hash and the computed hash indicates a read error—e.g., the information extracted from the nucleic acid molecules does not match the source data. To further improve tolerance to errors when reading data stored in nucleic acid molecules, an error protecting code such as a Reed-Solomon code can be applied to source data or the above-modified string of symbols that represents hashed source data. The Reed-Solomon code increases error tolerance, for both erroneous elements and element erasures, when reading data, as described in further detail below with reference to FIGS. 24-25.

Applying a uniform weight code to the data before writing it to nucleic acid molecules may also increase the efficiency of reading that data back from the nucleic acid molecules. Multiple identifier molecules may be located in a pool having solid, liquid, or powder form. For example, identifier molecules may be formed in separate compartments then the compartments may be consolidated to form the pool. A uniform weight code ensures that each compartment has a certain number of identifier molecules. The data may be separated into words and then translated to form codewords, in a manner that ensures that each resulting codeword has the same number of symbols of a particular type (e.g., when symbols are bits, all codewords could have the same number of bits having value 1), resulting in the codewords having the same “weight.” For example, in an NchooseK encoding scheme, each codeword may be represented by the identifiers formed in one compartment, and each compartment would contain exactly K identifier sequences of N possible sequences (note that the pool or compartment includes copies of individual identifier molecules, where each copy of identifier molecules has the same identifier sequence. As used herein, “a number of identifier sequences” or “a number of identifiers” in a pool or a compartment refers to a number of copies of individual identifier molecules, where each copy corresponds to the same identifier sequence). When sequencing that pool or compartment, if fewer than K identifier sequences are read (or observed) for the N possible sequences that represent a codeword, that would indicate that there is insufficient data to interpret the value of the codeword. We may refer to such an event as a codeword erasure. On the other hand, once K identifier sequences have been read (for N possible sequences that represent a codeword) during sequencing, the sequencing process can stop, which can decrease the amount of sequencing time and improve efficiency needed to interpret the codeword. In some examples, if more than K identifier sequences are read for N possible sequences that represent a codeword, then the codeword may be interpreted from the K identifier sequences with the highest copy numbers. In some examples, all combinations of K identifier sequences from the observed >K sequences may be considered to determine a more limited set of possible codeword values. The correct value may be determined in further downstream processing, for example, with hashing.

One way to improve efficiency in reading information from DNA involves using a data structure to hold the location of data blocks of data string. For example, a large data string may be separated and stored into two or more containers. To determine which container contains information a user wants to access, the system may access a B-tree or trie structure that holds the location (e.g., container number or placement). This allows a user to access the information he or she is looking for in an expedient manner—rather than reading the information in each of the containers containing the data string. Further, the information a user wants to access may only comprise a plurality of identifiers that is smaller than the total number of identifiers contained in a container. In such instances, it would be more efficient and costly to access and read only a small subset of possible identifiers comprising the target plurality rather than the entire space of possible identifiers that exist in the container. So the location information contained in the B-tree or trie structure may be further configured to contain information about the target plurality of identifiers in addition to the container.

The systems and methods described herein thus provide several opportunities to decrease the cost and increase the throughput of writing information into nucleic acid molecules. First, a set of components can be reused and recombined to write new packets of digital information. The expensive requirement to use base-by-base synthesis for each new write job is thus replaced by a one-time base-by-base synthesis cost that may be amortized over several write jobs (e.g., 224 30-base oligos at 10-μmol scale to write 860 terabit packets). Second, the encoded information is de-coupled from the sequences of DNA components, enabling the use of a large sequence design space that may be optimized for write, store, copy, query, and read operations. Third, the nucleic acid molecule encoding schemes described herein comprise enhanced error correction and provide optimized operation speed.

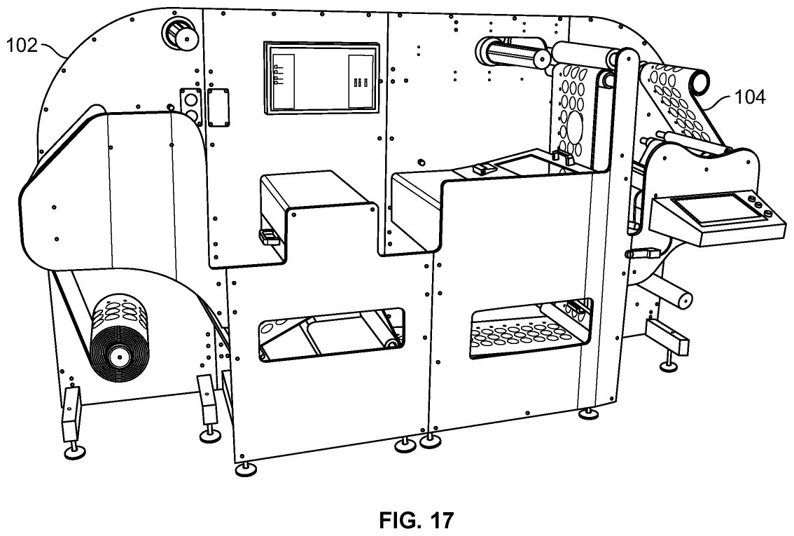

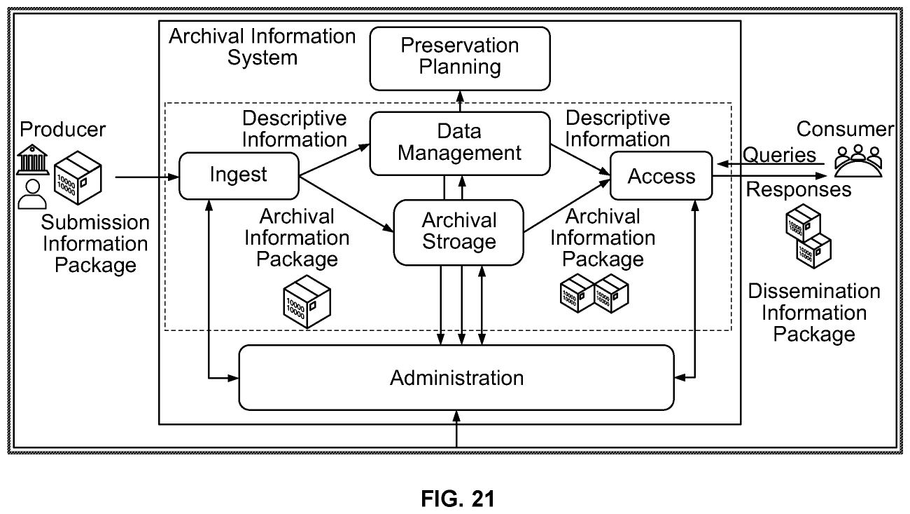

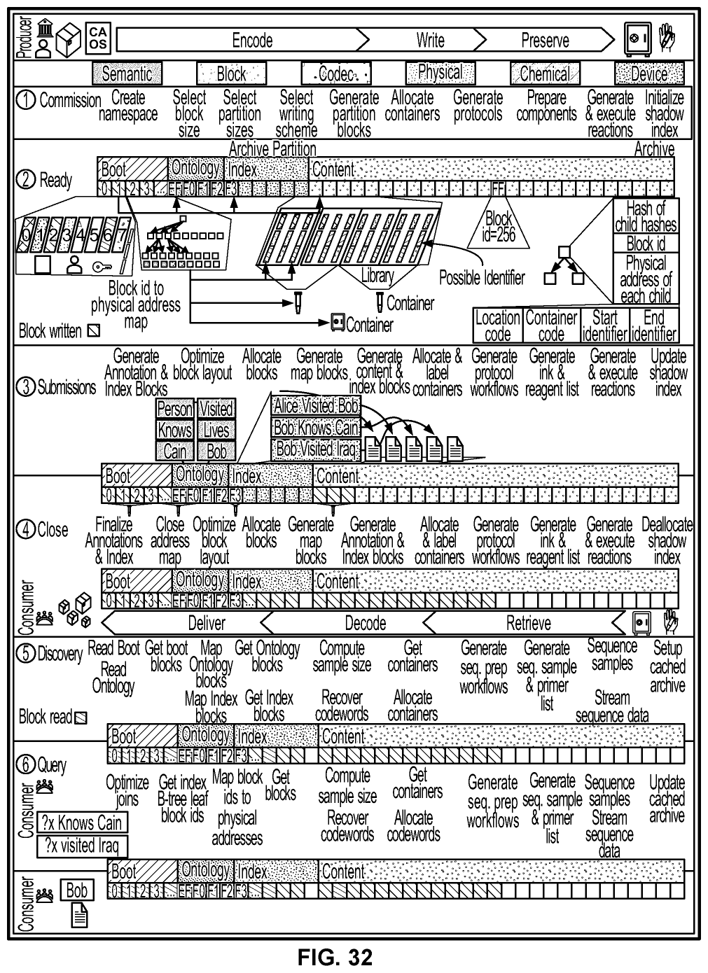

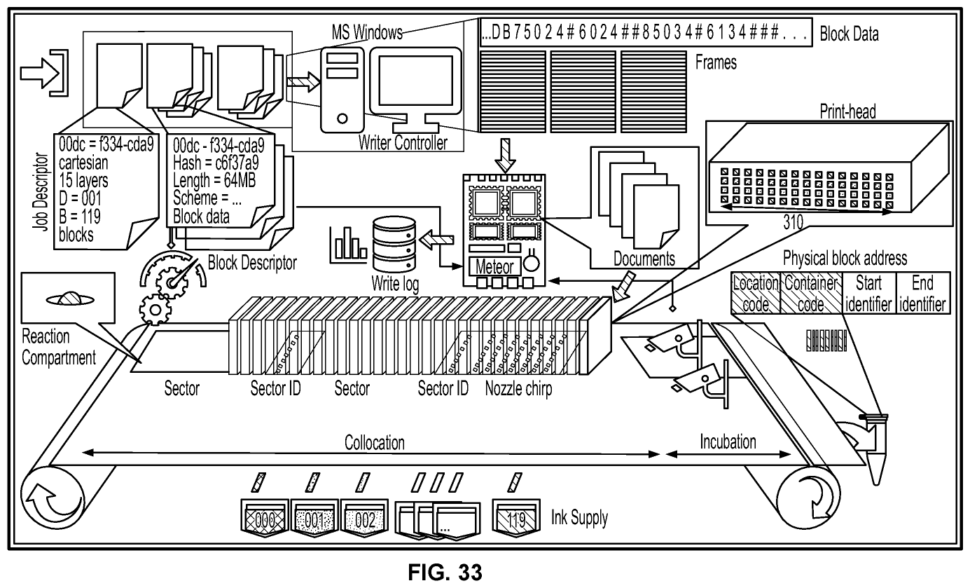

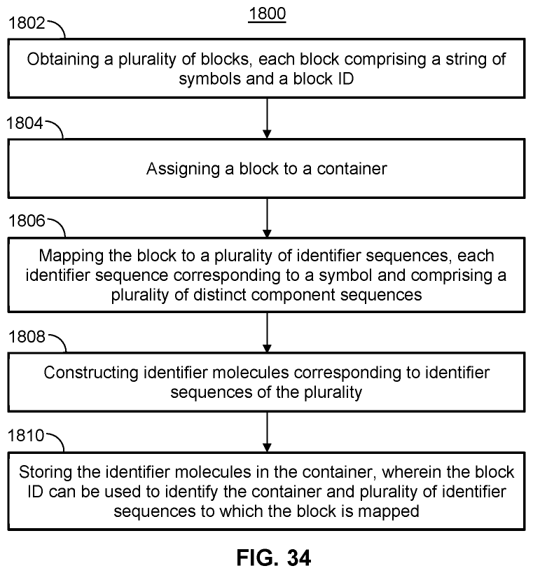

The following description starts with an overview of encoding data in nucleic acid molecules, followed by a description of writing and archival systems configured to print and store encoded nucleic acid molecules as described in relation to FIGS. 17-21. The present disclosure describes various encoding methods in relation to FIGS. 22-27, including schemes designed for error protection, efficient encoding, and “weight minimization”. The description then describes decoding systems and methods in FIGS. 28-31. The description introduces data structures that can be used in the retrieval of specific information from nucleic acid molecules in FIG. 34. FIG. 32 shows an overview of archival operations and FIG. 33 shows a system diagram of writing data to nucleic acid molecules.

Writing information to nucleic acid molecules using the methods described herein involve encoding a string of symbols as identifier sequences, where the position and value of each symbol is represented by an identifier sequence. In some implementations, each identifier molecule is comprised of ligated premade DNA component molecules that are ordered based on defined layers. Within each layer, several unique DNA component sequences can be selected to make an identifier sequence. The one-to-one mapping of a symbol to its corresponding identifier sequence is established by an identifier order, which is an efficiently computed function of its components. As a specific example, the set of available identifier sequences may include 15 layers, 14 layers of which each contain six unique DNA component sequences. The 15th layer may be a multiplex layer comprising 28 DNA component sequences (rather than six) which will also be incorporated. Thus, each identifier may contain 15 components (one component in each layer) in the full-length identifier nucleic acid molecule. During the writing process, the component molecules are assembled together in reaction compartments to form identifier molecules. In some implementations, multiple components from only the “multiplex layer” will be combined into the same reaction compartment.

As an example, to write one terabyte in 86400 seconds (24 hours), approximately 8E+11 identifier molecules may need to be assembled (assuming 10 bits of information encoded per identifier), or approximately 5.7E+10 droplet reaction compartments. Each reaction may assemble fourteen identifiers from a possible set of 28 identifiers. Fourteen components (one from each of the 14 layers each with six possible components) specify and assemble the “base” of the identifiers. A remaining fourteen components out of 28 possible components from the multiplex layer specify which fourteen identifiers (out of 28 possibilities) will be assembled. Thus, each reaction compartment may need 28 DNA components, plus ligase or other reaction mix.

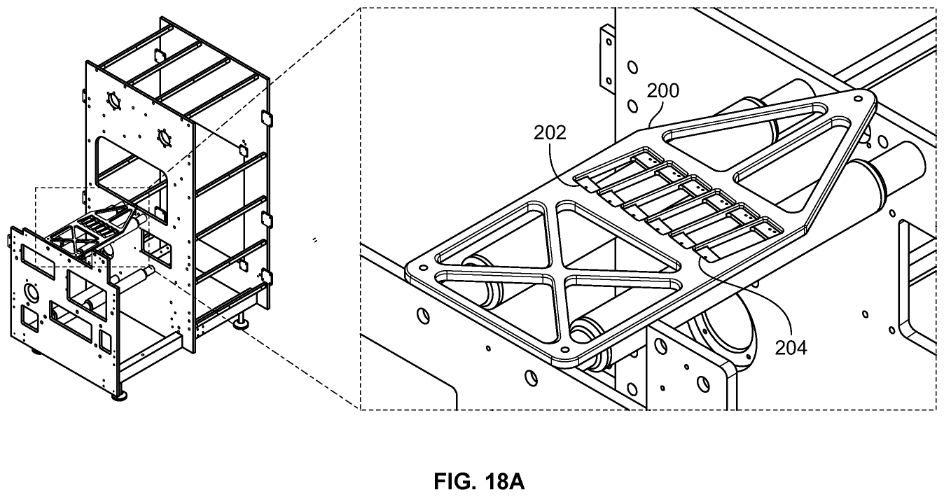

The methods described herein may be implemented using a writing system, as described below. The writing system may be a printer-finisher system such as that described in U.S. application Ser. No. 16/414,752 filed May 16, 2019, entitled Printer-Finisher System for Data Storage in DNA, which is hereby incorporated by reference. The writer system may dispense DNA components at discrete locations (e.g., reaction compartments) on a substrate, dispense ligation master mix, provide optimal conditions for the ligation reaction, and pool all of the DNA identifiers that comprise a library. The writing system may comprise four modular components a base instrument, a print engine, an incubator, and a pooling sub-system, as described below in relation to FIGS. 18-21.

The writing systems described herein may execute high-throughput, parallelized printing of ligation reactions for constructing identifiers. Reactions may be carried out in picoliter (pL)-scale droplets printed onto flexible sheets (also referred to as webbing or substrates) moving over rollers. The writing systems may incorporate technologies such as digital inkjet printing and web handling, using suitable off-the-shelf print heads, drivers, and machine infrastructure. The systems and methods described herein include optimization of factors such as web speed, print head dispense speed, droplet size, and ligation chemistry to achieve storage capacity and write throughput. To this end, and to ensure data tolerance to potential chemistry and hardware errors, the systems and methods described herein include configurations to encode the data and develop printing instructions, including specifications for how to partition DNA component sequences into layers and how many identifier molecules to construct in each printed reaction. For example, such configurations may include computer systems that communicate with the writing system and track its performance.

FIG. 17 shows a base module 102 of a writing system configured to execute methods described herein. Base module 102 contains the unwind/rewind components that move webbing 104 through the writing system, such that components may deposited thereon. Webbing 104 (also referred to as a substrate) is a material on to which droplet will be dispensed to form droplet reaction compartments. For example, the webbing may be a low binding plastic like polyethylene terephthalate (PET) or polypropylene. Base module 102 is a reel-to-reel machine. Base module 102 may, for example, be the Werosys Compact system that was developed for the research and label manufacturing industries.

The writing system also includes a print engine. The two main components of the print engine are the ink management system and print heads for droplet dispensing. The ink management system includes a vacuum pump, valving/tubing, and on-board software/electronics for local control of the vacuum pressure in the headspace above the liquid. For example, the ink management system may be a Megnajet system. The ink reservoir can be located up to 1 m away and may use a Meteor 4-color controller card.

FIG. 18A shows a print head mounting rack 200. Rack 200 holds up to six print heads in print head holding spots 202-204. Rack 200 is an interface between the print heads used to deposit components onto the webbing and the webbing passing through the print engine. Rack 200 holds the print heads close the webbing. For example, the print heads may be held approximately 0.5 mm, 1 mm, 2 mm, or 3 mm above the web. Rack 200 holds the print heads at a cant to appropriately line up print head nozzles for over-printing in reaction compartments because certain print heads have a misaligned nozzle arrangement that limits our ability to overprint droplets. The cant angle is a slight rotation from the normal vector of the nozzle plate so that the nozzles align to over-print droplets. The cant angle may be, for example, 1 degree, 2 degrees, 4 degrees, 8 degrees, 10 degrees, 20 degrees, or any suitable angle. To achieve an overprint of, for example, four different nozzle rows, the print head may rotated at an angle of 8.7°. The accuracy of droplet dispensing is driven by the desired spacing, the diameter of a droplet on the web, which is dependent on the contact angle, and by the repeatability of dispensing. These parameters can be met with available hardware components but may need some customization during development.

FIG. 18B shows four rows of print head nozzles 212, 214, 216, 218. Each of rows 212, 214, 216, 218 may dispense a different component. Substrate 222 (which extends diagonally upward and to the right from the line pointed at by arrow 219) is moved linearly under the print head with nozzles 212, 214, 216, 218. Because of the 8.7 degree rotation of the print head, a coordinate 224 on substrate 222 will pass directly beneath nozzles in rows 212, 214, 216, 218 along line 217 such that each nozzle may deposit a component on coordinate 224.

The print heads can dispense multiple “colors” per print head (four) which allow for overprinting. The droplet volume dispensed by each nozzle of the print head may be 1 pL, 2 pL, 3 pL, 7 pL, 10 pL, 20 pL, or any other suitable amount. In some implementations, the volume of the droplets may be adjusted. Flexibility in droplet volume is helpful because this parameter affects evaporation rate and ligation incubation conditions. Additives may be added to the component inks to facilitate compatibility with the print heads. For example, solutes like tris may be added to increase conductivity. As an example, humectants or surfactants (e.g. glycerol) may be added to improve ejection quality and print head nozzle lifetime.

In some implementations, the print heads are MEMS (micro-electro-mechanical system) devices. For example, the print head may be a Ricoh MH5420 print head. Print heads are selected to avoid the risk and uncertainty associated with many thermal print heads, which may compromise the integrity of DNA identifiers. The print heads are capable of fast, low volume, aqueous-compatible and drop-addressable piezoelectric dispensing. The print heads may include a stainless steel nozzle plate.

Nozzle clogging is a common failure mode for print heads. For example, a stopped print head is at risk of blockage, drying out, and thus needing recirculation. For this reason, the writing system allows allow print heads to be moved from the writing system for purging and wiping and then replaced while maintaining registration.

To help repeatability of printing, the writing system may optimize droplet morphology, volume, and speed. The solution in the print heads may comprise water-Tris solution that contains dye for visibility. For example, the dye may be bromophenol blue. The solution used in the print heads varies from traditional print head inks in that it has low viscosity and high surface tension. The solution is essentially water and will have low viscosity at ˜1 cP, compared to ideal ink of 10-11 cP. Because the solution is essentially water, it will have a high surface tension at 72 mN/m, compared to ideal ink of 32 mN/m

With optimal waveforms, droplet repeatability may be verified by measuring droplet shape, volume and speed. Individual droplet volume may be verified dispensing millions of drops into a mineral oil vessel and measuring the change in mass of the vessel. To avoid liquid evaporation, the droplets may be dispensed into the oil such that the aqueous droplets submerge. Droplet shape and speed may be measured using a “drop watch” system using a CCD camera to capture droplet dispensing in-flight.

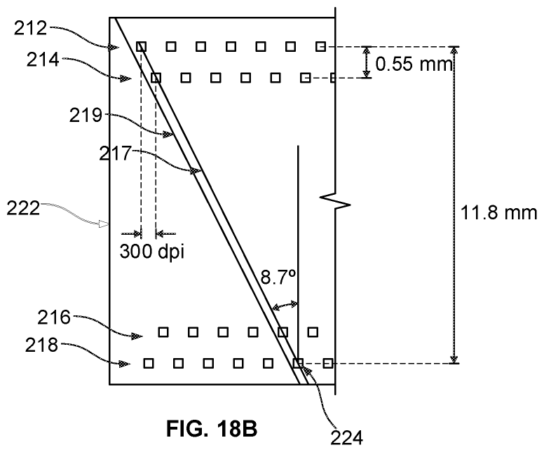

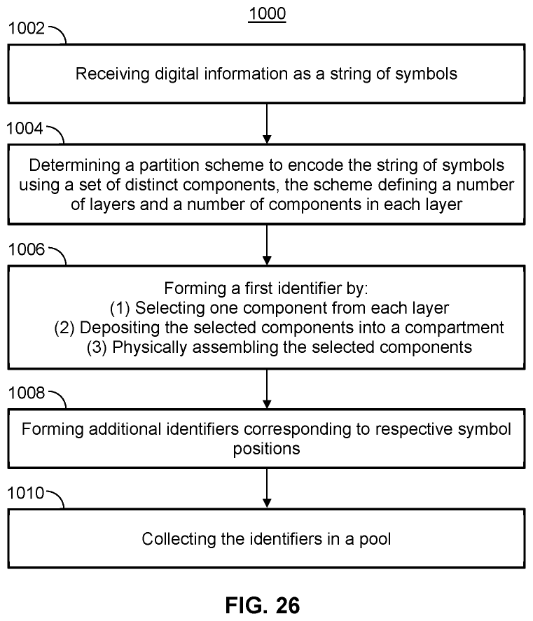

FIG. 19 shows a diagram of dispensing solutions into reaction compartments according to identifier sequences constructed from a trie. Each layer of the trie represents a layer of the identifier, and each edge of each trie layer represents a component within corresponding identifier layer. The final layer of the trie may represent the multiplex layer of an identifier. String of symbols 300 represents a set of codewords to be stored in DNA identifier nucleic acid molecules. A codeword is a string of symbols that represents a specific string of symbols from a source alphabet, called a source word. A code maps source words to codewords in a process known as encoding. Droplets 304 are dispensed into individual reaction compartments (e.g., compartment 306). String of symbols 300 is encoded by constructing identifiers that are represented by the trie paths connecting the root of the tree (not shown) to the leaves of the final (multiplex) layer 302 that point to symbol values of ‘1’. As shown in FIG. 19 and as will be described further below, particularly in relation to FIG. 27, the codeword's “weight” may be evenly distributed—i.e., string of symbols 300 is divided such that each string of five bits is encoded in a reaction compartment, and each string of five bits has exactly three ‘1’ values so each compartment receives three components from the multiplex layer to form three identifiers. For example, reaction compartment 306 will contain identifiers with a unique combination of components from the base layers (the components that comprise the path of the trie up to the multiplex layer—components 7, B, D, . . . ), and components 0, 2, and 4 from the multiplex layer, respectively, as shown in the multiplex layer and as corresponding to the positions of the string of symbols “10101”. For each of the ‘1’ values, the corresponding identifier molecule for that symbol position (comprised of the components that make up the path leading to it) will be deposited into the reaction compartment.

After the print heads have deposited components into the reaction compartments, the reaction compartments are moved from the print engine to the incubator module. The incubator is critical for the ligation reaction because it controls the temperature required for optimal ligation efficiency and humidity needed for preventing droplet evaporation. The incubator module uses a series of rollers to keep large portions of webbing (e.g., 10 m, 20 m, 40 m, 100 m, or any suitable length) in the chamber for the duration of the ligation reaction. The number and position of the rollers allows changes in incubation time or webbing speed. While in the incubator module, the concentration of solutes within the droplet in the reaction compartment need to remain constant to retain maximal ligation efficiency. For this reason, the primary function of the incubator is to maintain a level of humidity that will minimize volume loss due to evaporation. Factors affecting the evaporation rates of droplets are (1) a humectant (likely to be glycerol) may be required within the ligase liquid, in order to prevent full evaporation of the droplet in a time much shorter (<1 s) than the required ligation time and (2) the concentration of glycerol within the ligase strongly affects the required humidity levels and humidity tolerances.

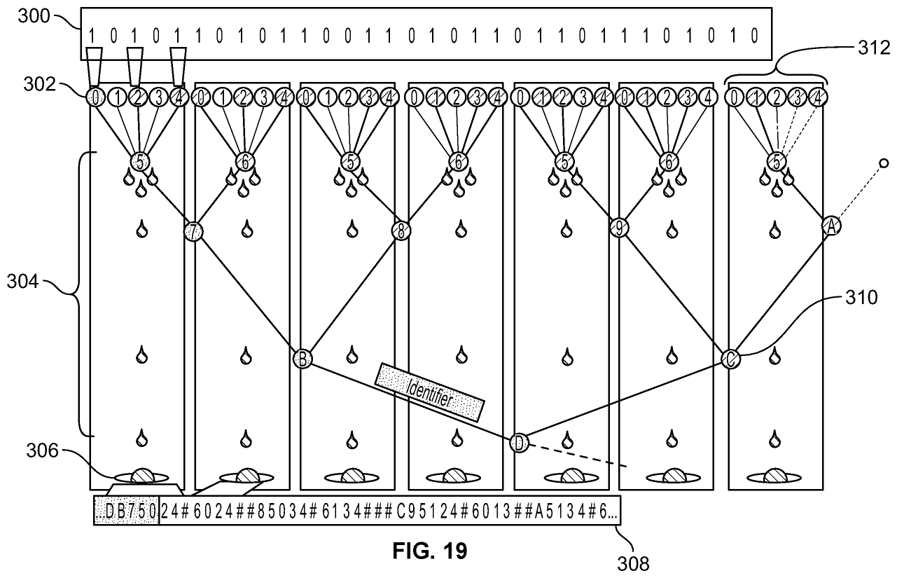

FIG. 20 shows a pooling sub-system using spray wash 414 and immersion in pooling buffer 408 to remove printed droplets from webbing 416. The pooling sub-system removes DNA molecules from moving web 416 and transfers it to a DNA binding column. The webbing 416 passes over rollers 402, 404,406. The DNA molecules are removed from the webbing 416 using a combination of two mechanisms (1) spray wash 414 of the moving web using the pooling buffer and (2) immersion of the moving web into the pooling buffer 408 within the pooling chamber. In some implementations, only one of the two mechanisms is used, or the mechanisms are used in combination with other DNA molecule removing means.

After removal from webbing 416, the DNA molecules are passed through a binding column to collect the full-length DNA identifiers 410. The column 410 is then removed from the writing system for down-stream processing, comprising DNA elution and collection within a suitable vessel for storage.

The systems and methods described herein provide a fully automatable workflow based around nanopore sequencing to decode molecular data. The workflow includes validating a physical DNA storage system, developing PCR-based data access methods, improving nanopore sequencing technologies (with altered sample preparation, purposefully designed DNA component sequences, and chemically modified nucleic acids), and optimizing sequencing systems and workflows for large-scale parallel sequencing on multiple devices. DNA-based information produced using the methods described herein is uniquely suited to retrieval by strategically optimized nanopore (or electronic channel) sequencing methods. A key hurdle for nanopore sequencers has been achieving slow enough DNA translocation and narrow enough pores to sequence with single-base resolution. The DNA components encoded by the methods described here may be designed to simultaneously boost sequence signal of each component molecule and increase the discrimination between component sequences. Together with the option of incorporating chemically modified bases to enhance signal to noise, these features allow nanopore technologies to achieve reproducible TB-scale recovery of artificial DNA information. The system and methods described herein provide for the development of organization and storage systems for DNA, the development of a protocol for accessing a DNA library, the modification of sample preparation protocols for improved reading capacity of encoded DNA, and the advancement of nanopore technology to increase reading capacity of sequencing instruments.

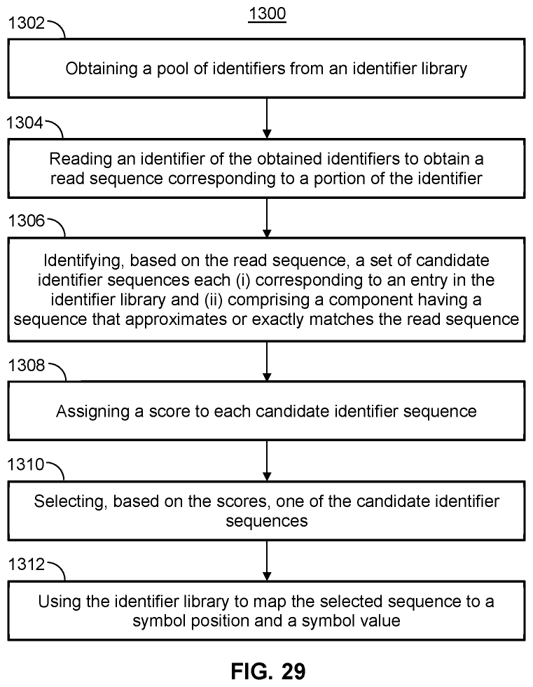

The output from the writing process of a string of symbols, as described above, is a library of encoded DNA (identifiers) that may require long-term storage and infrequent access. The produced pool of encoded DNA may contains hundreds of thousands of molecules of each identifier sequence. In terms of grams, the total amount of material produced may be in microgram quantities. The pool may be amplified with PCR to ensure enough material exists for redundancy, archiving, and accessing, as described below in relation to FIG. 30.

After amplification, the pool may be allocated into multiple containers and stored in different locations. The pool may be stored in a range of nucleic acid storage and archival systems. For example, DNA may be stored in Eppendorf tubes, in a freezer, cryo-preserved in liquid nitrogen, or stored in Tris-EDTA. Shelf-life of DNA assessed by reading material subjected to accelerated stability conditions such as different temperatures. The systems and methods described herein may include an automated sample management system that allows for both long-term storage and random access of stored DNA.

In some implementations, an operating system (OS) may be capable of coordinating writing, reading, discoverable querying of archives scalable to Exabyte sizes, or any combination thereof. Specifically, in some implementations, the OS aims to enable the reading and writing of a tree of semantically annotated and indexed blocks via a codec optimized for the read/write platform described above. The OS includes a translation stack that can include an ingest API, as well as modules for organizing and formatting data for long-term yet granular data querying and discovery. These aspects of the OS can be broadly suited for any writing, reading, or access method. Other aspects of the OS can be designed to specifically optimize methods for writing, accessing, and reading information. These include modules for compressing and error-protecting data, as well as modules for configuring and sending data to the writing systems described above. Though data written in DNA molecules with the above methods will be readable with any sequencer, specific reading methods are described below. The OS may also include automation software and workflows that mediate the handling of DNA-based information between the writer and reader; for example, by allocating DNA to, accessing DNA from, and replenishing DNA in a system of storage containers capable of supporting an Exabyte of information.

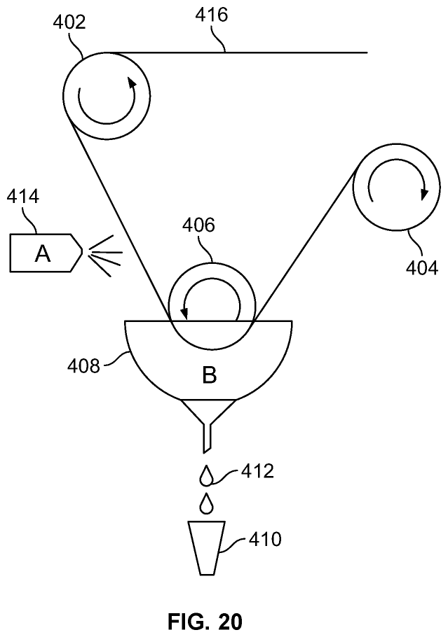

FIG. 21 shows an archival information system for the OS. The central module comprises ingestion, data management, access, and archival storage modules. The data management and archival storage modules communicate with the ingestion and access modules. Descriptive information is passed from the ingestion module to the data management module. Descriptive information is then passed from the data management module to the access module. Archival information package is passed from the ingestion module to the archival storage module and then to the access module. The central module takes in information from a producer, such as a submission information package, via the ingestion module. The central module also communications with the consumer, via the access module, by fielding queries and sending responses. For example, the queries and responses may be held in a dissemination information package. The data management and archival storage units also interact with the preservation planning and administration modules.

FIG. 22 shows a layered organization of the capabilities managed by the OS organized into seven functional layers. Each layer draws on the services offered by the layer below. The seven layers translate into six areas of development involving design and construction of:
(1) Codec: an encoder/decoder pipeline with writer-specific optimizations
(2) Chemistry Interface: a translator from bit operations to chemical operations
(3) Automation Interface: interfaces and translators to automation devices
(4) Block Abstraction: a block-based interface & supporting core data structures
(5) Search & Indexing: infrastructure for semantic annotation and indexing
(6) Archival Application: an archival application demonstrating the OS
   Benefits of the encoding schemes and OS described herein include the ability to select an encoding scheme optimized for writing speed, writing cost, reading cost, or access cost; the ability to optimize the mapping of index data to blocks to minimize decoded footprint; the ability to manipulate information at all scales from large blocks to single bits and model data structures natively; and tight integration with current archival standards and practices enabling archival, querying, and reasoning over data and relationships.

The codec functions as the encoder/decoder for information. Because layers above need it and layers below cannot be meaningfully tested without it, the proper operation of the codec is highly important. The codec receives a source bit stream and is charged with translating it into a form suitable for writing using chemical methods. As shown in FIG. 22, the Codec includes three layers: a fixity layer, a redundancy layer, and a combinatorial layer that handle the encoding process.

In the fixity layer, the source bit stream is divided into packets, where all packets are of a fixed size. Packets may be processed independently and serve as a unit for parallel processing. Packets are composed of one or more blocks. A block is the smallest unit of allocation in the archive, and the archive's combinatorial space is divided into a series of contiguous strings of bits called blocks. The fixity layer is responsible for computing a block hash using a standard cryptographic hashing algorithm such as MD5, SHA-224, SHA-256, SHA-384, SHA-512, SHA-512/224, or SHA-512/256 and this hash is included in a parent block. When a block is decoded, its integrity may be checked by re-computing its hash and checking it via the parent block.

In the redundancy layer, the hashed block is passed on to the redundancy layer where up to two, three, or more error protection techniques are applied. Because the susceptibility to noise of certain writing systems may be unknown, a high redundancy convolution code may be used. Errors caused during writing may be primarily of two types: (1) due to a missing identifier, for example because of deteriorating print head nozzles or low efficiency assembly reactions, or (2) due to assembly of unintended identifiers, for example because of dispense splatter or contamination among adjacent reactions. A writing system's imaging-based quality control methods may mitigate errors due to print head clogging and missing identifiers. To correct errors, a block is divided into slices (e.g., 223 bytes long in a typical configuration) and a Reed-Solomon code using an arithmetic field of 255 symbols is computed for each slice, resulting in error protection bytes (e.g., 32) capable of correcting byte-errors (e.g., 16 bytes) (where each byte in error may have any number of bit errors). These error protection parameters are configurable, are written to the archive, and are configured to tolerate, for example, a writing system error rate of 104 errors per codeword. As defined above, codeword is a string of symbols that represents a specific string of symbols from a source alphabet, called a source word. A code maps source words to codewords during the process known as encoding.

Assuming a scheme where a source word comprises three source bytes, a protected slice of 255 bytes will map to 255/3=85 codewords. Assuming independent errors, the probability that more than 16 bytes will be in error is the probability that more than five codewords will be in errors, which is approximately 4.3×10⁻¹⁶. By changing the field size or the error protection bytes computed, this probability can be lowered as needed. A writing system with precision printing heads, such as that described above, may be able to comply with this expected error rate, but the codec is capable of handling higher protection rates if needed, albeit at a higher computing cost. Additionally, a larger field (e.g. 65,535) could confer higher protection. To mitigate the impact of unintended identifier molecules, the redundancy layer may also introduce an optional interleaver that permutes the order of the bits so that source bits protected by the same set of error protection bytes do not end up in adjacent reaction compartments and thus do not become susceptible to larger burst errors than may be correctible.

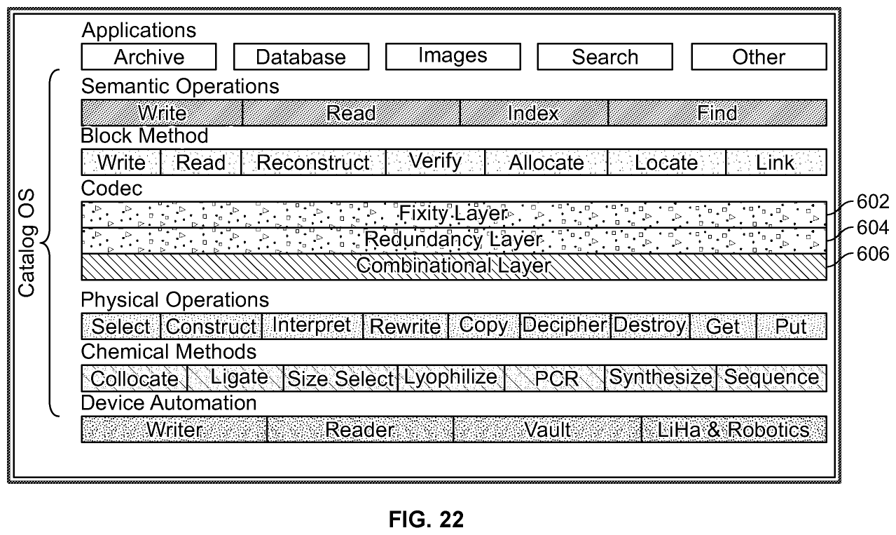

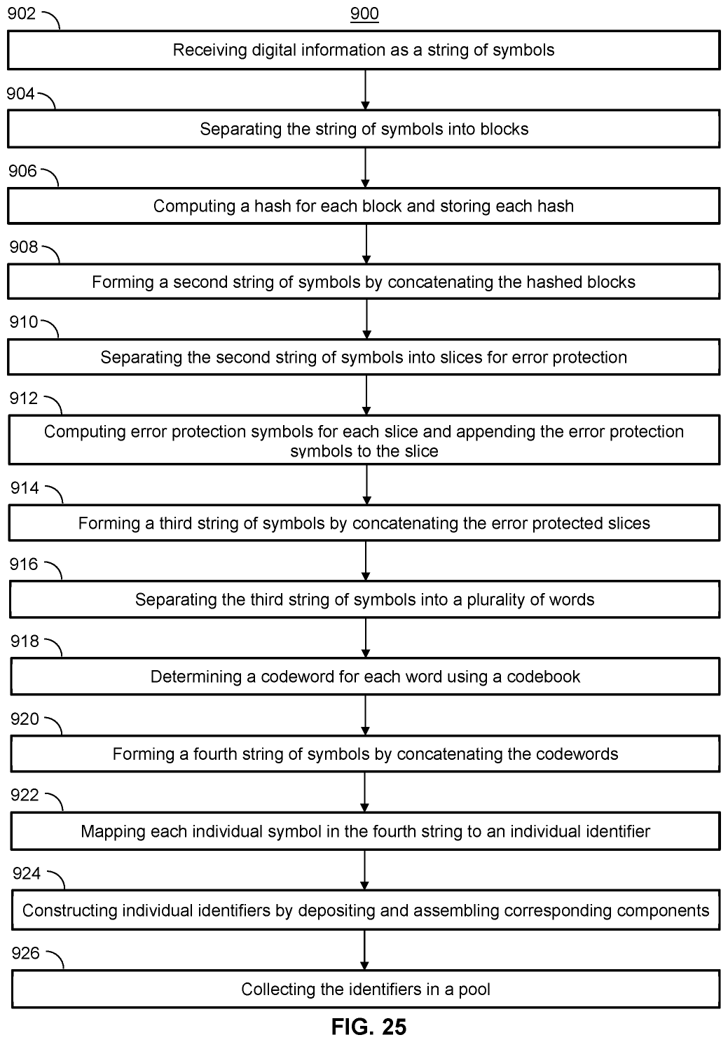

Symbols, such as the error-protected symbols formed from the methods described in relation to FIGS. 24-25, may be mapped from source words composed of source symbols (e.g., a series of bits) to codewords from a number of combinatorial methods for encoding information in nucleic acid libraries. In some implementations, this encoding is performed or orchestrated by the codec. An example of a particular encoding scheme is shown in FIG. 23. FIG. 24 shows a system diagram of a Layered Cartesian Product combinatorial constructor (LCPCC) for designing and ordering identifiers. The product constructor has three layers (M=3) and a component library of eight component sequences (C=8). A combinatorial partition scheme with {3,3,2} component sequences per layer is shown. The space of all possible identifier sequences forms a combinatorial space. As will be described in further detail below in relation to FIG. 26, the total number of combinatorial objects constructible from the component library, which may be referred to as the span of a combinatorial scheme, determines the length of the bit stream writable to a single pool of identifiers. The span of this particular scheme is 18. The combinatorial objects are ordered in the combinatorial space lexicographically by extending a ranking on the component sequences to the identifiers constructed from them. This ordering information is implicit in the identifier, identifies its position in the combinatorial space, and may be used to identify the position of the symbol encoded by the identifier in the source symbol stream. For example, the LCPCC may be used to encode a binary alphabet, and define the symbol encoded by a constructed identifier to be “1.” The “0” symbol may be represented by not constructing the corresponding identifier. A source bit stream may be encoded by constructing a specific set of identifiers (i.e., an identifier library) unique to that bit stream.

FIGS. 24-25 show flowcharts 800, 900 for storing digital information into nucleic acids with error protection. At step 802, information is received as a string of symbols of length L. As described above, the symbols may be bits, bytes, a bit string of any length, alphanumeric characters, a character string of any length or any other suitable symbol. In some implementations, the string of symbols is converted into a bit stream. For example, the string of symbols may consist of six symbols “LETTER”. The string of alphanumeric characters “LETTER” may be converted to binary, resulting in 48 bits (“01001100 01000101 01010100 01010100 01000101 01010010”). In this example, L would be equal to 48. In some implementations, the information in the string of symbols is received separately—i.e., symbols may be received individually or any combination. The symbols or groups of symbols may then be concatenated or otherwise combined to form a string of symbols. For example, ten blocks of 8 bits may be received individually and then concatenated together to form a string of symbols 80 bits long.

Flowchart 800 includes three stages of encoding the received information: (1) hashing, as explained in relation to steps 804-808; (2) adding error protection symbols, as explained in relation to steps 810-814; and (3) determining codewords, as explained in relation to steps 816-820. While FIG. 24 displays all three of these stages, the stages may be performed separately or in any combination thereof. For example, steps 804-814 (hashing and adding error protection symbols) may be bypassed, such that flowchart 800 proceeds from step 802 directly to step 816 (the start of the codeword stage). The stages may be performed in any order.

The hashing stage begins at step 804, where the string of symbols is separated into blocks. In some implementations, each block is of the same length B, where B is equal to L (the length of the string of symbols) divided by the number of blocks. As an example, the string of symbols may be 1,000 bits. The 1,000 bits may be separated into any length of blocks, such as five blocks each consisting of 200 bits; 100 blocks each consisting of 10 bits; 10 blocks each consisting of 100 bits, or any such combination. In some implementations, the blocks are not of equal length. For example, for a string of 1,000 bits, blocks may consist of 500 bits; block₂ of 100 bits; block₃ of 300 bits; and block₄ of 100 bits. In some embodiments, blocks may be padded with arbitrary symbols to reach a target length.

At step 806, a hash of length H is computed for each block. In some implementations, the hash is computed using one of MD5, SHA-224, SHA-256, SHA-384, SHA-512, SHA-512/224, or SHA-512/256. Each computed hash is appended to the corresponding block to form a hashed block. This allows string of symbols and the hashes to be stored together in nucleic acids. An alternative implementation that includes storing the hashes separately from the string of symbols is described below in relation to FIG. 25. If each block is of length B, after appending the hash, the hashed blocks are of length H plus B. For example, the string of symbols may consist 1,000 bits that are then separated into ten blocks of 100 bits each (B=100). The hash of each block may be 10 bits long (H=10). After appending, the hashed blocks then consist of 110 bits (the original 100 bits plus the 10-bit hash).

At step 808, the hashed blocks are concatenated to form a second string of symbols of length L2. In the example above, where each hashed block of the ten hashed blocks comprises 110 bits, the string of symbols would be equal to 1,100 bits (the original 1,000 bits received in step 802 plus 100 hash bits computed in step 806).

At step 810, the second string of symbols is separated into slices. In some implementations, each slice is of the same length S, where S is equal to L2 (the length of the second string of symbols formed in step 808 from the hashed blocks) divided by the number of slices. As an example, the second string of symbols may consist of 1,100 bits. The 1,100 bits may be separated into any length of slices, such as ten slices each consisting of 110 bits; one hundred slices each consisting of 11 bits; five slices of 220 bits, or any such combination. In some implementations, the slices are not of equal length. For example, for a string of 1,100 bits, slice₁ may consist of 500 bits; slice₂ of 100 bits; slice₃ of 300 bits; and slice₄ of 200 bits. In some implementations, the hashing stage (steps 804-808) is bypassed. Thus, the second string of symbols references in step 810 would be equal to the string of symbols received in step 802. In some implementations, the blocks are never concatenated back together to form L2, but rather each block is processed into slices separately.

At step 812, error protection symbols are computed for each slice. Any number of error protection symbols may be computed for each slice. At step 812, the error protection symbols are appended to the slice for which they were computed, thereby forming error-protected slices. In some implementations, the same number P of error protection symbols are computed for each slice, such that the each error-protected slice is S plus P bits long. For example, if the second string consists of concatenated blocks that are each 1,100 bytes (1000 source bytes plus a 100-byte hash), they can each be divided into five slices consisting of 220 bytes. Then 40 bytes of error protection can appended to each slice resulting in error-protected slices consisting of 260 bytes each.

In some implementations, the error protection symbols are determined using a Reed-Solomon code. Reed-Solomon codes are block-based error correcting codes. If the P error protection symbols (or bytes) are computed using a Reed-Solomon code,

$$\frac{P}{2}$$

erroneous symbols (or bytes) can be tolerated in a protected slice. For example, if P is equal to 40 bytes for a 260-byte protected slice, 20 of the 260 bytes may be incorrect without negatively affecting the processing of those 260 bytes. If the P error protection bytes are computed using a Reed-Solomon code, P erased bytes can be tolerated. For example, if P is equal to 40 bytes for a 260-byte protected slice, 40 of the 260 bytes may be erased without negatively affecting the processing of those 260 bytes.

At step 814, the error-protected slices are concatenated to form a third string of symbols having length L3. In the example above, where each error-protected slice of the five error-protected slices per block comprises 260 bytes, the third string of symbols would consist of 1,300 bytes per block (the original 1,000 bytes received in step 802 plus 100 hash bytes computed in step 806 plus 200 error protection bytes total determined in step 812). In some implementations, flowchart 800 proceeds from step 808 to step 816, bypassing the error protection stage (steps 810-814). Thus, the third string of symbols references in step 814 would be equal to the second string of symbols formed in step 808. In some implementations, both the hashing and error protection stages are bypassed, such that flowchart 800 proceeds from step 802 directly to step 816. In this case, the third string of symbols would be equal to the string of symbols received in step 802. In some implementations, the third string of symbols is computed and processed separately for each block.

At step 816, the third string is separated into a plurality of words, each word having length W. For example each word may be eight bits long. At step 818, a codeword is determined for each word using at least one codebook. In some implementations, each codeword is an exact match of the respective word (i.e., nothing changes between the third and fourth strings of symbols). In some implementations, however, the codewords may be different from their respective words. For example, the codewords can be a different length than the words.

Codewords may be optimized for chemical and instrument conditions during encoding or decoding. As described above, the presence of an identifier may indicate a ‘1’ in a certain symbol position, while the absence of an identifier for that position would indicate a ‘0’. In some implementations, determining the codewords comprises applying a uniform weight code (e.g., as described in relation to FIG. 27) to the third string of symbols to ensure every codeword has the same number of ‘1’s (i.e., identifiers to be constructed in step 824). Thus, for the example above with W=8, a codeword must be chosen that can encode eight bits. This effectively adds additional bits so that each codeword can have an equal “weight” (number of identifiers to be constructed).

As described in relation to FIG. 23, the codewords may be determined from a combinatorial space in an NchooseK scheme. In an Nchoosek scheme, for every N ordered identifiers, k identifiers are constructed. For example, in an 11choose4 scheme, for every 11 identifiers, exactly four are constructed. The representative codeword would have length N=11 bits and have weight k=4. An 11choose4 scheme provides enough bits to encode 8 bits in each codeword because Log₂(11choose4)>8.

At step 820, a fourth string of symbols having length L4 is formed by concatenating the codewords. The fourth string of symbols will comprise

$$\frac{L 3 * N}{W}$$

symbols. For example, tor a string of 1300 bytes per block (e.g., the third string of symbols formed in step 814) if each word is 8 bits and an 11choose4 scheme is used, L4 would equal 1787.5 bytes per block, or 14300 bits if each byte is eight bits. In some embodiments, the fourth string of symbols is computed and processed separately for each block.

At step 822, each symbol in the fourth string is mapped to an individual identifier sequence. The mapping step 822 produces a scheme for printing the digital information into nucleic acids with error protection. An individual identifier nucleic acid molecule of the plurality of identifier nucleic acid molecules corresponds to an individual symbol in the fourth string of symbols. An individual identifier comprises a corresponding plurality of components, wherein each component in the plurality of components comprises a distinct nucleic acid sequence. For example, the components may be selected from M layers as described above.

At step 824, the individual identifier molecules are constructed by depositing (or co-locating) and assembling corresponding components. For example, the printer/finisher system described above in relation to FIGS. 17-21 may be used to construct individual identifiers by depositing and assembling corresponding components in a compartment. In some implementations, dispensing, using a plurality of print heads, a plurality of solutions comprising a plurality of components onto a coordinate on a substrate. A reaction mix may be dispensed onto the coordinate on the substrate to physically link the plurality of components, provide a condition necessary to physically link the plurality of components, or both.

Based on the codeword stage, a set of printer instructions may be developed that are then sent to a printer-finisher system. The printer instructions may be configured to reduce the possibility of printing errors or increase printing efficiency. For example, the codeword stage may be designed to distribute the identifiers such that each compartment in the plurality of compartments contains the same number of copies of each identifier nucleic acid sequence to within a statistical certainty, thus providing uniform abundance of identifiers across compartments. To provide additional error protection, in some implementations, codewords are permuted or interleaved (with respect to one another) before being sent to the writing instrument, such that identifier nucleic acid molecules that represent adjacent symbols in the string of symbols are not constructed in adjacent compartments. This reduces the chance that burst errors in the writing instrument results in uncorrectable errors or erasures. For example, this reduces the chances that printing mistakes may cause undetectable errors due to streaks of printing onto the wrong coordinate or bleeding between compartments. Alternatively, the error protection may be computed on disparate symbols rather than adjacent symbols to reduce the chance that burst errors in the writing instruments results in uncorrectable errors or erasures

At step 826, the individual identifiers are collected in a pool. For example, a pool can hold hundreds of identifiers corresponding to hundreds of symbols encoded in steps 802-822. In some implementations, a presence or absence of an identifier in the pool is representative of the symbol value of the corresponding respective symbol position within the string of symbols.

Flowchart 900 follows the same steps with the exception of storing the hashes rather than appending them. Steps 902, 904, 908, 910, 912, 914, 916, 918, 920, 922, 924, and 926 are equivalent to steps 802, 804, 808, 810, 812, 814, 816, 818, 820, 822, 824, and 826, respectively. In step 906, hashes are computed for each block but not appended; rather, the hashes are stored separately. For example, the hashes may be stored on a hard drive in order for the hashes to be accessed faster or more easily than the blocks in nucleic acids. The hashes could be stored in nucleic acid molecules, a magnetic storage device, a flash memory device, cloud storage, or any other suitable location.

When encoding a string of symbols, the number of distinct identifier sequences that can be constructed depend on the parameters of the encoding scheme used and the string of symbols to be encoded. For a given string of symbols, it may be advantageous to generate an optimized scheme that minimizes key resources (e.g., the number of layers or components used to build the identifiers). A set of C components may be partitioned in B(C) distinct ways, where B(C) is the C^{th} Bell number, and increases factorially with C. If the L layers contain c₁, c₂, . . . , c_{L} components respectively, then the total number of distinct identifiers constructible is Πᵢ₌₁^{L} cᵢ, with Πᵢ₌₁^{L} cᵢ=C. The total number of combinatorial objects constructible from the component library, which may be referred to as the span of a combinatorial scheme (as noted in relation to FIG. 23), determines the length of the bit stream writable to a single pool of identifiers. Therefore, for a given bit stream length and component library size, a partition scheme with a span large enough to encode the bit stream, while minimizing the number of layers, may be beneficial. Each additional layer imposes additional efficiency and time constraints on the chemistry; therefore a partition scheme with fewer layers is preferable. An example of a partition scheme to minimize layers is described below in relation to FIG. 26. A branch-and-bound search over the space of partitions possible from the prime factorization of the bit stream length.

The codec or any encoding system may use several strategies to compute a set of multiplexable reactions. First, a strategy borrowed from multi-valued logic synthesis may treat all or parts of a string of symbols, such as a bit stream, as a Boolean function and attempt to extract a minimal representation of the function using heuristics. State of the art logic synthesis tools have been shown to be able to handle functions with ˜10⁶ row truth tables. If the source stream is already entropy compressed, then this approach may fail because succinct representations may be difficult to find. For compressed streams, a local greedy approach is to divide the stream into reaction words of Z adjacent identifier sequences, and use a component partition scheme that has at least Z component sequences in the multiplexing layer. This forces Z identifiers to share the same prefix, and facilitates assembling them in a single reaction compartment. (For example, Z=2 in FIG. 23.) Inversely, another performance measure of interest is the number of bits encoded per reaction. Minimizing reaction compartments increases the number of symbols written in a single reaction, thus minimizing the write time and the total substrate length needed by the writing system to write a given string of symbols.

Another key resource is the number of identifiers that can be constructed to encode a source bit. By default, an LCPCC scheme encodes each symbol position of a string of symbols with a unique identifier sequence. If the string of symbols is a bitstream, where a ‘1’ is indicated by the presence of an identifier and a ‘0’ is represented by its absence, the number of identifiers that can be assembled to write the bit stream is proportional to the number of bit values in the source bit stream. Unlike in silico compression where bit stream length is a key measure, here it is the weight of the bit stream—the number of “1” bit values—that may define the writing time or reaction compartments needed.

Assembling identifiers (e.g., the identifiers described in step 1008 of FIG. 26) may require performing a chemical reaction in a compartment where all the necessary components and reagents are collocated at the time of the reaction. The number of such compartments needed is a critical resource that may be minimized. One strategy to minimizing reaction compartment count is to execute multiple reactions in each compartment. Not all reactions, however, may be mutually combinable. The LCPCC (described above in relation to FIG. 23) generates a combinatorial space such that adjacent identifier sequences share a common prefix of component sequences. For the example in FIG. 23, identifier sequences 0 and 1 share a common component sequence prefix—identifier sequence 0 has component sequences 0, 3, 6 and identifier sequence 1 has component sequences 0, 3, 7. This implies that the construction of identifiers 0 and 1 may be executed in a single reaction compartment in which four components are collocated: 0, 3, 6, and 7, because components 6 and 7, by construction, are mutually inert. This strategy of executing multiple reactions in a single compartment may be referred to as multiplexing, and the layer from which multiple components are taken, the multiplexing layer.

FIG. 26 shows a flowchart 1000 outlining the steps for encoding digital information into nucleic acids with a partition scheme. The partition scheme may be designed with the intention of encoding the digital information in nucleic acids under restraints, such as writing hardware configurations (e.g., the number of inks available in a printer). Flowchart 1000 begins at step 1002. Like steps 802 and 902 of FIGS. 24-25 described above, at step 1002 digital information is received as a string of symbols having length L. In some implementations, an additional step may be incorporated in flowchart 1000 to translate the string of symbols to a string of bits of length B.

In order to encode within the given restraints, at step 1004 a partition scheme is determined. The partition scheme defines a set of rules to encode the string of symbols using a set of C distinct component sequences. Specifically, the partition scheme defines a number of M layers within which to arrange the C distinct component sequences, and defines component sequences numbers in each layer, such that there are cᵢ component sequences in the i^{th} layer. In some implementations, the number of component sequences in each layer is non-uniform (i.e., c₁ is not equal to c₂, etc.). The number of layers and number of component sequences may be configured to minimize the number of layers necessary to encode the string of symbols, thereby simplifying the chemistry of forming identifier molecules while maintaining enough identifier sequence possibilities to encode the entirety of the digital information. To ensure enough identifier sequence possibilities to encode the string of symbols, a product of the component sequence numbers cᵢ (Πᵢ₌₁^{M} cᵢ) must be greater than or equal to the length (as measured in bits) of the string of symbols and a sum of the component sequence numbers cᵢ (Πᵢ₌₁^{M} cᵢ) must be less than or equal to the number C of distinct component sequences. In some implementations, the identifiers are representative of a subset of a combinatorial space of possible identifier sequences, each including one component from each of the M layers. As a simple example, if L equals 1,000 bits (e.g., 1,000 bits in a bit stream received in step 1002) and C equals 70 (e.g., 70 printer inks available to be printed), three layers (M=3) with 10 component sequences per layer could be used to encode the data. However, to best capitalize on the available 70 component sequences, it may be more efficient to encode the data using two layers (M=2), with 50 component sequences in the first layer and 20 component sequences in the second layer.

In some implementations, if the string of symbols has been translated to a string of symbols of length B, the product of the component sequence numbers cᵢ must be greater than or equal to the length of the string of symbols of length B, converted to bits. For example, if the translated string of symbols is “LETTER”, B equals 6 but the equivalent string of bits consists of 48 bits (B equals 48) if each character is encoded by 8 bits. Thus, the number of M layers and C component sequences necessary to encode “LETTER” must be such that Πᵢ₌₁^{M} cᵢ≥48.

At step 1006, a first identifier is formed, for example with a printer-finisher system, by (i) selecting one component from each of the M layers, (ii) depositing the M selected components into a compartment, and (iii) physically assembling the selected components. In some implementations, the selected components are assembled by ligation. In some examples, the M layers are associated with different priority levels. For example, the first layer may have a highest priority and the second layer may have a second highest priority.

At step 1008, additional identifiers are formed. The additional identifiers correspond to respective symbol positions in the string of symbols that represents the digital information to be encoded. Each symbol position within the string of symbols may have a corresponding different identifier. Once the necessary amount of identifiers are formed, the identifiers are collected in a pool in step 1010. In some aspects, information is read from nucleic acid sequences. In some implementations, a pool of identifiers is obtained. The identifiers in the pool store digital information from a string of symbols of length L. The pool of identifiers corresponds to a subset of identifier sequences in an identifier library that is capable of encoding any string of symbols having length L. Each individual identifier in the pool corresponds to a symbol value and a symbol position in the string of symbols. Each individual identifier comprises a plurality of components and is thus an instance of a specific sequence. In some implementations, the pool comprises gene-, peptide-, or RNA-encoding DNA.

FIG. 27 demonstrates a uniform weight code to translate each word into a codeword with fixed weight. Uniform weight coding can improve decoding and simplify a writing system's configuration. One approach to this is to construct a complete bipartite graph of all possible source words and their mapping to all possible codewords. Each edge of the complete bipartite graph is weighted by the number of ‘1’ bit values introduced by the codeword. By choosing a minimal weight matching, a problem for which linear programming-based polynomial time algorithms exist, one could find a mapping that minimizes the bit stream weight, effectively “compressing” the total number of identifier sequences needed to write it. Such a code may be referred to as a weight minimizing code. The number of “1” bit values may be minimized by recoding the source bit stream using longer codewords. In FIG. 27, the minimum weight of a codeword of length five necessary to encode a word of three bits, is weight two. However, because three bit words will on average have a weight of 1.5, this codeword scheme, though minimal in weight for codewords of length 5, actually increases the total weight of the message. If, for example, the codewords were extended to length eight, then there could be a codeword weight of one to encode three bits. In this example, the overall weight of the message would be lessened. Weight minimizing techniques may be used to reduce the number of identifier sequences needed per source bit.

Reading an identifier library by sequencing involves sampling from a distribution of multiple copies (multiple nucleic acid molecules) of distinct identifier sequences. Non-uniformities in enrichment of identifiers can make sampling lower copy identifiers difficult, pushing the need for larger samples. Because the writing systems described herein assemble multiple identifiers in reaction compartments via multiplexing, and because the number of identifier sequences in each reaction compartment is defined by the source bit stream, the enrichment of each identifier in the final library could vary. One approach to mitigating this problem is to recode the source bit stream using a uniform weight code, one in which every codeword contains a fixed constant number of “1” values, as shown in FIG. 27. Consequently, each reaction compartment assembles a fixed number of identifiers and thus the variance in enrichment is minimized without modification to the writing system. Moreover, by choosing the codeword length judiciously, a uniform weight code could also minimize the weight of the bit stream. For example, the set of 37.4×10⁶ 28-bit codewords of a fixed weight of 13 can encode all 25-bit source words, yielding 25/13=1.92 bits/identifier. As another example, the set of 64.5×10⁶ 32-bit codewords of a fixed weight of 10 can encode all 25-bit source words, yielding 25/10=2.5 bits/identifier.

An additional benefit of uniform weight coding is that when a library is read, each codeword decoded is expected to contain a known fixed number of “1” values which enables the use of more robust decoding techniques. Pushing this idea to the extreme, a long codeword, for example 1024 bits, could span multiple reactions, yet may require assembling only a single identifier encoding 10 bits; this can lead to extreme “weight compression” enabling high encoding rates due to the need to construct very few identifiers, and high decoding rates due to higher bits encoded per identifier.

Because the recoding techniques described above are applied in the combinatorial layer after classical redundancy bytes have been computed, they can have an impact on the error protection performance. For example, when a 25-bit source word is mapped to a 28-bit codeword of uniform weight 13, the mapping may not be isometric: a 1-codeword error can now cause a multi-byte error in the source word. Extreme weight minimization may also affect the SNR (signal-to-noise ratio) of an encoded bit because the minimum distance of the code is reduced. To investigate and remedy these issues, weight minimizing uniform weight codes may be used that include source words may be embedded into codewords near-isometrically. This may result in the use of longer codewords, but also offer better error performance. To generate an encoding scheme that co-optimizes against all these constraints, tools may minimize layers, maximize span, minimize reaction compartments, minimize weight, and find a code that uses constant weight codewords that preserve error protection performance.

A throughput of roughly 1 Mb/s may be achievable per CPU-thread for the encoder as described above. As an example, a rate of 93 Mb/s (1 TB/day) may be achievable using four instances of 32 CPUs, −10 TB of temporary disk space, and −3 TB of outgoing bandwidth, which may be amortized over 1080 jobs. The cost of producing this encoded information may be roughly halved if similar infrastructure were locally connected to the writing system, saving the cost of outgoing bandwidth and assuming ingest was free. Using GPUs or server-less cloud functions could reduce this cost further, at the expense of software refactoring and platform dependence.

Recovering a source bit stream from an archive follows a roughly inverse process to the one described above. FIG. 28 shows a system diagram with the basic steps of decoding a set of identifiers. The system obtains a pool of the subset of identifiers of interest. The goal is to reconstruct a set of identifier sequences with the highest likelihood to have produced the sequences observed in an adequate sampling of the pool. Using a simulation model, a sample size estimate is first computed for the identifier sub-library to be decoded, given a small acceptable probability of incomplete sampling. Using the compute sample size, the identifier library is sampled and sequenced to obtain a stream or collection of observed sequences.

Consider an observed sequence s. From the representation information stored the archive described above it is known that the sequence comes from a LCPCC identifier library with L layers, where the jth component of layer i contains component sequence cᵢⱼ. Therefore, the observed sequence s is first compared with component sequences cᵢⱼ for all j. If an exact match is found, then the unmatched suffix of s undergoes this same process now with i=2. If no exact match is found, then a fast approximate matching score is computed for any prefix of s and component sequences c₁ⱼ using an approximate string matching technique (ASM) or alignment technique, or an n-gram approach. ASM (approximate string matching) methods may be evaluated to determine their suitability to “online” matching of streaming sequence data, as is expected from a nanopore sequencing device, described below.

For a component sequence of length l_{c}, only the variable segment of the component sequence need be identified. The variable segment of the component sequence may be as small as lc/3, unlike bit-by-bit writing schemes that endeavor to decode every base. This process is repeated with the unprocessed suffix of s and results in an L-partite graph with vertices weighted by a match score. The top weighted paths correspond to candidate identifier sequences, and each candidate has a score. Identifier sequences belong to an ordered combinatorial space, so each candidate identifier sequence corresponds to a symbol in the encoded stream. Some candidate identifier sequences may contradict the codeword rules; for example, a codeword may be of a fixed weight. These candidates may be eliminated based on low scores or saved as an alternative set of candidates. Finally, a path through top ranking candidates is constructed to obtain a candidate sequence of codewords. This sequence of codewords is then checked against fixity data and corrected if possible using error correction symbols. In cases of extreme noise or error, the technique could backtrack and choose an alternative path through candidate identifiers to search for the correct sequence of candidates.

Once codewords are obtained, they are mapped back to source words using an implicit Lehmer codebook to obtain the error-protected source blocks. These blocks are then decoded and checked to verify fixity. If errors are found, error protection symbols are used to correct them if possible and the source blocks are recovered. The source blocks are appropriately assembled into a source bit stream and handed off to the block layer for query response assembly, delivery, and caching.

FIG. 29 shows a flowchart 1300 outlining the steps for reading digital information stored in nucleic acid sequences. At step 1302, a pool of identifiers is obtained (e.g., via the methods described in relation to FIG. 30). The identifiers in the pool store digital information from a string of symbols of length L. The pool of identifiers corresponds to a subset of identifier sequences in an identifier library that is capable of encoding any string of symbols having length L. Each individual identifier in the pool corresponds to a symbol value and a symbol position in the string of symbols. Each individual identifier comprises a plurality of components and is thus an instance of a specific sequence.

Between steps 1302 and 1304, the identifier may be processed in various ways. In some implementations, the identifier is ligated to a second identifier. In some implementations, one strand of the identifier is degraded. For example, a strand-specific exonuclease may be used to selectively degrade one strand of the identifier.

At step 1304, at least one of the obtained identifiers is read to obtain a read sequence corresponding to a portion of the at least one identifier. Step 1304 may be accomplished by any sequencing technique, such as chemical sequencing, chain termination sequencing, shotgun sequencing, bridge PCR sequencing, single-molecule real-time sequencing, ion semiconductor sequencing, pyrosequencing, sequencing by synthesis, combinatorial probe anchor synthesis sequencing, sequencing by ligation, nanopore sequencing, nanochannel sequencing, massively parallel signature sequencing, Polony sequencing, DNA nanoball sequencing, single molecule fluorescent sequencing, tunneling current sequencing, sequencing by hybridization, mass spectrometry sequencing, microfluidic sequencing, transmission electron microscopy sequencing, RNA polymerase sequencing, or in vitro virus sequencing. Sequencing a pool of nucleic acids, identifiers in this case, yields a read sequence for the whole pool; however, it is not known how each identifier of the pool maps to the read sequence. Sequencing methods are prone to single-base errors, further hindering the matching of identifiers to the read sequence.

In some implementations, step 1304 includes nanopore sequencing. An electric field is applied to an electrolytic solution and at least one nanopore channel. In some implementations, the at least one nanopore channel is formed within a solid-state membrane. In some implementations, the nanopore channels are formed from alpha-hemolysin (αHL) or Mycobacterium smegmatis porin A (MspA). During nanopore sequencing, the identifier is translocated through the at least one nanopore channel, while impedance across the channel is measured. Each component in the identifier has a corresponding unique impedance signature along the length of the component sequence, thus allowing the components in the read sequence to be determined by comparing measured impedance values to the unique impedance signature.

In some implementations, when step 1304 includes nanopore sequencing, the applied electric field generates a differential potential greater than or equal to 100 mV. This high differential potential enables the identifier to be passed through the nanopore channels at a relatively high rate. For example, translocation of the identifier may occur at a rate great than 1,000 bases per second. In particular, the translocation rate may be 1,000,000 bases per second.

In some implementations, when step 1304 includes nanopore sequencing, an agent is bound to the identifier before translocating. For example, the agent may be a chemical moiety, a protein, an enzyme, a base analogue, a conjugated nucleic acid, a nucleic acid with a hairpin, or a methyl group. In some implementations, if the agent is a chemical moiety, an enzyme, such as methyltransferase, binds the chemical moiety to the at least one identifier nucleic acid molecule. In some implementations, if the agent is a base analogue and the agent is bound using an enzyme, such as a polymerase, the enzyme binds the base analogue to the at least one identifier nucleic acid molecule during replication.

The agent is associated with an agent signature that may be used to help determine sequences in the identifier during reading. Binding the agent to the at least one identifier nucleic acid molecule occurs at a known location on a component of the identifier, such that the agent signature at the known location results in a known shift in impedance value during translocation. The presence of the agent may thus create an exaggerated “profile” for the identifier, thereby increasing the signal-to-noise ratio during reading. This may allow the translocation speed to be increased while maintaining accuracy during reading. In particular, the presence of the agent on the at least one identifier may allow for a first maximum translocation rate that achieves a desired level of accuracy that is faster than a second maximum translocation rate that achieves the desired level of accuracy in the absence of the agent on the at least one nucleic acid molecule. Another way to increase signal-to-noise ration during reading includes replicating the identifier such that it comprises modified bases or base analogues. This may done separately or in addition to binding an agent to the identifier.

Steps 1306, 1308, 1310, and 1312 describe a method of matching the read sequence to a known set of identifier sequences (i.e., the identifier library). In step 1306, the read sequence is used to identify a set of candidate identifier sequences from the identifier library that have a component sequence that approximates or exactly matches the read sequence. As an example, the read sequence (which may or may not correctly match the identifier) may be CAGCTG. The set of candidate identifier sequences may comprise an exact match (CAGCTG) as well as other potential matches that are similar to the read sequence, such as identifier sequences that differ by a certain number of bases (e.g., 1, 2, 3, 10, 20, 100, etc.). For example, the set of candidate identifier sequences may also include CAGATG, AAGCTA, and CACGTG. For ease of reference, the “incorrect” nucleotides (i.e., that do not match the read sequence in the example) are underlined.

In some implementations, the identifiers are encoded such that each identifier is associated with a reading error tolerance (for example by ensuring a minimum hamming or Levenshtein distance between components of the same layer). A permissive reading error tolerance may be used to increase the rate at which the identifier is read. Another way to increase read speeds includes reading a subset of the identifier. In some implementations, the identifier includes M components corresponding to M layers (as described above in relation to FIG. 26). In some implementations, reading the identifier includes reading only N of the M components where N is less than M. For example, only the first two layers out of five layers may be read. This can increase how many identifiers are read in a given amount of time. This can be helpful when a subset of the data encoded in the pool needs to be accessed. For example, if the first layer always indicates a certain meaningful value, certain useful identifiers can be identified by only accessing the first layer. Or, for example, if all accessed identifiers have the same components in the first four layers, then those four layers need not be read.

In step 1308, each candidate identifier sequence is assigned a score associated with how similar the component sequence of each candidate identifier sequence is to the read sequence. The better the candidate identifier sequence matches the read sequence, the lower (or higher) the score may be. The scores may be computed in a variety of ways including a least distance algorithm, a percent match, or any other suitable algorithm. As an example, for the read sequence CAGCTG, a candidate sequence CAGCTG may have a score of zero, while a candidate sequence CAGATG may have a score of one because the fourth base of the candidate sequence does not match the read sequence. The score may depend on the number of bases that are incorrect and/or the placement of incorrect bases. For example, a candidate with two incorrect nucleotides adjacent one another (CACGTG) may have a lower score than a candidate with two incorrect nucleotides that are not adjacent (AAGCTA).

The set of scores guides the decision in step 1310 to select one of the candidate identifier sequences as a potential match to the identifier that was read (or observed) in step 1304, thereby mitigating the effect of single-base sequencing errors. For example, the candidate sequence with the lowest score may be selected because it is the closest potential match to the read sequence. At step 1312, the selected candidate identifier sequence is then mapped to a symbol position and symbol value using the identifier library. In some implementations, if step 1304 includes nanopore sequencing and an agent has been bound to the identifier, determining the sequence in the identifier includes comparing measured impedance values during translocation to the agent signature.

In some implementations, steps 1306, 1308, 1310, and 1312 are iterated until the desired digital information is completely accessed from the pool (or multiple pools) of identifiers. In some implementations, a decoded string of symbols is determined and tested for accuracy. Specifically, a hash of a portion of the decoded string of symbols may be calculated then compared to a hash associated with a corresponding portion of the string of symbols obtained in step 1302. The hash may be stored as a plurality identifiers in the pool (and subsequently be read via the steps of FIG. 29 above) as described in relation to FIG. 24, or may be stored remotely, as described in relation to FIG. 25. The hash may be calculated using MD5, SHA-224, SHA-256, SHA-384, SHA-512, SHA-512/224, or SHA-512/256 or any other suitable algorithm. A mismatch between the read out or original hash and the computed hash indicates there was an read error in reading the data—e.g., the information extracted from the nucleic acid molecules does not match the source data. Based on whether the calculated hash matches the read out original hash, the portion of the decoded string of symbols may be verified as a match to the portion of the string of symbols obtained in step 1302. If the hashes do not match (or if it is determined the decoded string does not match the obtained string through any other means), a different candidate identifier sequence (e.g., of the set of candidate identifier sequences from step 1306) may be selected (e.g., as in step 1310 described above). As identifiers are decoded and verified, a computer system may track this information and use a machine learning technique to increase a likelihood that the decoded string of symbols matches the string of symbols. The error-tolerant method disclosed by FIG. 29 thus set a basis for making unconventional improvements to sequencing techniques, for example running nanopore sequencing at a substantially large applied voltage, as described above.

In terms of grams, the total amount of material in a given pool may be in microgram quantities. To accurately read the molecules in the pool, it may be amplified with PCR to ensure enough material exists for redundancy, archiving, and accessing. The components on each edge of the identifiers can be designed to have common primer binding sites so that entire identifier libraries can be replicated exponentially in one PCR reaction. etc. The amplifications process may include two primary steps. In the first amplification step with (A) primers, the desired data block is selected and enriched; in the second with (B) primers, amplicons are prepared for sequencing. The first step (A) is intended to select specific targets, using the unique primers and only a few PCR cycles, and the second step (B) is intended to then amplify the target sequences of the specific targets, not including the primers, to generate a large amount of molecules for sequencing. With this nested approach for amplification, the number and identity of identifiers accessed is adjusted and the sequencing burden is reduced. In some implementations, amplification steps are limited to 7 cycles or less to reduce amplification bias in the libraries and to maintain uniformity of sequence abundance. Primer combinations may be validated with identifiers to demonstrate efficiency and uniformity of the amplification process. Initial optimization will be measured by qPCR. The level of product purity may be determined by using PCR cleanup requirements (e.g., ExoSAP-IT, ThermoFisher) by measuring both qPCR amplification efficiency and sequencing the presence of partial sequencing products. It may also be advantageous to selectively enrich the molecules with tags to increase the speed of accuracy of reading. Similar to how the nucleic acid molecules can be tagged for better signal resolution in nanopore sequencing, each component or identifier may be tagged with a specific probe/adapter that allows for other selection techniques like protein- or magnet-based selection protocols. Examples of methods of enrichment, include i) streptavidin coated magnetic beads, ii) Ampure XP size selection, iii) specific primer capture by Watson-Crick bait sequences.

Moreover, a nested PCR-based, chemical random access method can be used to efficiently sub-library of identifiers for reading. FIG. 30 illustrates an example of a nested PCR amplification scheme for accessing a sub-library of identifiers from an archived DNA library. A sub-library may be any group of identifiers that one wishes to access, such as a data block, several data blocks, a single identifier, several identifiers, etc. In the first amplification step with (A) primers that bind components on the edge layers of the identifiers, a group of identifiers comprising the desired data block is selected and enriched. This process is repeated with (B) primers that bind components on the next two layers inward and therefore further reduced the group of identifiers to a smaller sample that comprise the desired data block. This process may be repeated until the retrieved (or accessed, or selected) group of identifiers is only or nearly only comprised of all identifiers that represent the desired data block, thus enabling the data block to be read back efficiently with sequencing. In the final round of nested PCR, the identifiers may be simultaneously prepared for sequencing. With this nested approach for amplification, the number and identity of identifiers accessed is adjusted and the sequencing burden is reduced. In some implementations, amplification steps are limited to seven cycles or less to reduce amplification bias in the libraries and to maintain uniformity of sequence abundance. Primer combinations may be validated with identifiers to demonstrate efficiency and uniformity of the amplification process. As described below, this access method may be used in conjunction with data structures to retrieve blocks of data by their associated block ID. As an alternative to nested PCR, affinity tagged probes that uniquely bind particular components may be used in a nested fashion to access a particular sub-library of identifiers (that represent a data block, for example) for reading.

Reading the identifier nucleic acid molecules in 1304 may be accomplished via nanopore sequencing. Nanopore sequencing provides advantages due to size and scalability. Nanopore sequencing involves applying an electric field to an electrolytic solution and nanopores. Under the applied voltage, nucleic acid molecules pass through the nanopores, interrupting the flow of the electrolytic solution and causing a measurable impedance. Each nucleotide can be correlated to a unique impedance value such that a whole sequence corresponding to a nucleic acid molecule can be obtained by processing an impedance dataset. The nanopore can be formed by a channel in solid-state substrate or by a protein. The protein may be embedded in a lipid membrane or a solid-state substrate such as metal, metal alloy, and polymer-based substrates, and common nanopore proteins include alpha-hemolysin (αHL) and Mycobacterium smegmatis porn A (MspA). For a point of reference, Oxford Nanopore's PromethION system is approximately 1.5 square feet in size and is able to sequence 12 Tb (12.0E+12 base pairs) in 48 hours. It is important to note, that because the writing scheme described herein uses premade DNA components that are verified to have high fidelity, any sequence error is likely only introduced during sample preparation and sequencing. Further, single base resolution may not be needed to identify components present in identifiers. For these reasons, improvements to sequencing speed are enabled by adjusting sample preparation protocols and implementing compression techniques (at least for digital information not already compressed).

The standard template preparation scheme for Oxford nanopore sequencing involves ligation of adapter protein complexes to nucleic acid molecules. Some adapter proteins act as a hydrophobic tether allowing for the nucleic acid molecules to target the lipid bilayer reducing the time nanopores are unoccupied. Another protein, or motor, such as α-hemolysin separates the double-stranded nucleic acid molecules so that a single-strand enters the nanopore. This motor then helps ratchets the single-stranded nucleic acid molecules through the nanopore. This protocol is entirely compatible with the identifier libraries and amplicon enrichment plan described herein.

To increase sequencing efficiency, it may be advantageous to optimize nanopore sequencing by physically concatenating identifiers in amplified sub-libraries. Nanopore sequencing may require target nucleic acid molecules to find nanopores which contributes to reading speed. In order to increase read time identifiers may be physically concatenated into longer molecules by ligation. By increasing the length of sequenced molecules from less than 500 bp to 5,000 bp (or greater), pore occupancy may be maximized.

The speed that nucleic acid molecules translocate through the nanopore may be increased. Current nanopore sequencing instruments translocate nucleic acid molecules through nanopores at a rate of 500 base pairs per second. Establishing a differential membrane potential (e.g., greater than 100 mV) across a nanopore membrane translocates nucleic acid molecules at a higher rate (e.g., ˜1,000,000 bases per second). For most sequencing applications, a rate on the order of hundreds of thousands is too rapid and protein “motors” can be used to ratchet nucleic acid molecules through the pore in order for a single distinct base to be detected. Thus running nanopore sequencing without a “motor” may need generating single-stranded nucleic acid molecule input and amplification of nucleic acid molecule signal. Several methods exist for asymmetric PCR that achieve greater than 50% single-stranded nucleic acid molecules from a reaction. By adjusting primer-melting temperature, amplification primers can be designed to drive the reaction into linear amplification of one strand. Alternatively, strand-specific exonucleases, such as Lambda exonuclease, can be used to bind specifically to 5′ phosphorylated nucleic acid molecule strands and selectively degrade one strand of the duplex. The protocol may provide greater than 90% production of single-strand molecules.

Regarding signal amplification, nucleic acid molecules can be modified with agents to enhance the signal to noise ratio, essentially creating a “super signature”. For example, agents can be small molecules, chemical groups, base analogues, enzymes, proteins, protein complexes, peptides, or amino acids. One method for nanopore signal enhancement, mTAG (methyltransferase-directed transfer of activated groups), uses a methyltransferase to add a chemical group, like biotinylated S-adenosyl-L-methionine cofactor analogue, to the N6 atom of the adenine base in a sequence motif. If the agent is a base analogue, it can be added to the identifier molecule through PCR in which the base analogue is included in reaction mix and is incorporated into a complementary strand bound to a single strand of the identifier molecule during replication. The new hybrid of identifier molecule with base analogue can then be sequenced, and the base analogue can improve signal to noise ratio in a sequencing readout.

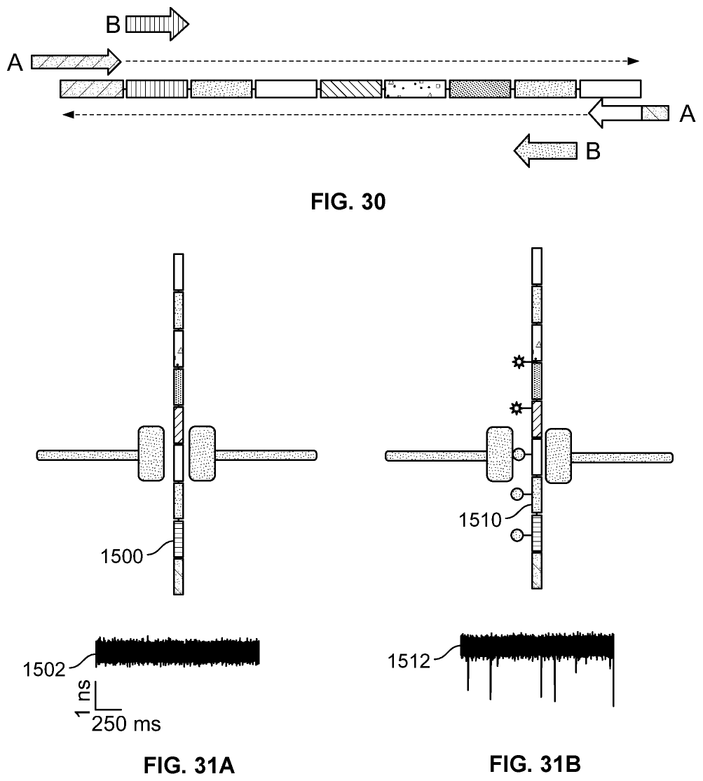

FIGS. 31A-B show DNA identifiers with signal events. In particular, FIGS. 31A-B show how modifying DNA with agents reduces conductance resulting in an increase of signal, thereby increasing the signal to noise ratio (SNR) associated with a sequencing event. Compared with unmodified DNA identifiers as shown in FIG. 31A, DNA identifiers with larger chemical structures as shown in FIG. 31B reduce conductance enhancing sequence signal. Graphs 1502, 1512 show signals (e.g., representative signal events from solid-state pores) from sequencing identifiers 1500 and 1510 respectively. Single and multiple modified bases can be used to create complex signal signatures defined by the type of modifications, number of modifications, and spacing between modifications. As shown, graph 1512 showing the signal of a modified identifier read shows a higher SNR than graph 1502 showing the signal of the unmodified identifier read.

In some implementations, a protein motor may be used to translocate nucleic acid molecules through the nanopore. A protein may be selected to increase the speed of translocation with the protein motor. For example, the translocation may be on the order of 1,000; 10,000; 100,000; or 1,000,000 base per second. Current motor proteins may be optimized to perform better at increased speeds. For example, published helicase variants for motor speed include synthesizing multiple variant construct (using commercial DNA synthesis vendors).

The core decoding effort comprises signal decoding and error recovery. As an example, suppose that the archive is written as 10 pools, each pool written with a component library of 113 component sequences partitioned into 17 sets of five component sequences each and one set of 28 component sequences. Each 25-bit source word is mapped to a 28-bit codeword of weight 14 identifier sequences. The span of such a combinatorial space is 21.4×10¹² identifiers per pool, with the size of any data encoding library being roughly 5.82×10¹² identifiers with 4.16×10¹¹ reactions. Assuming uniform enrichment of each identifier, and setting the incomplete sampling probability to 10⁻⁶, a sample of size 44×the size of the pool of 5.82×10¹² identifiers is needed. Thus, sequencing a single pool will result in 256×10¹² reads. Assuming each component is 30 bases long, each read will be 540 bases in length, resulting in a sequence stream of approximate length 34.6 PB (petabytes). Instead, if the codeword weight is reduced to four, so that each 14-bit source word is mapped to a 28-bit codeword of weight 4, then the span and the number of components that may be required remain unchanged, whereas the number of reactions increases to 2.97×10¹². Each identifier encodes 3.5 bits instead of 1.79 bits in the previous scheme, and the total reads are halved to 128×10¹² resulting in stream of length 17.2 PB. Using the 34.6 PB estimate, a stream of about 34.6×10¹⁵ bytes can be processed in the span of 24 hours, which may require a throughput of 3.2×10¹² b/s. Graphics Processing Units (GPUs), like the Nvidia GeForce GTX Titan X GPU with 12 GB of memory connected to a high performance Xeon CPU, may be used for approximate string matching achieving between 0.35×10¹² and 1×10¹² b/s depending on text and pattern length, and allowed edit distance. Using 10 instances of Nvidia Tesla V100, and a GPU comparable to the GeForce Titan X offered by a cloud provider, a higher signal decoding throughput may be achieved.

Assuming a writing error rate of 104 codewords, and the encoding scheme described above, we expect to see roughly 25 erroneous bits every 250000 bits, or every 122 255-byte slices. Assuming independent uniformly random errors, we thus expect a bit-error every four 255-byte slices. Thus, at the assumed error rate, at least 75% of all slices decoded would be error free. If decoding an erroneous slice takes three times as long as encoding it and decoding an error-free slice takes a third of the time as encoding it, the total decoding time works out to be roughly equal to the encoding time.

The signal decoding GPU setup may be cloud-based. From this, and not double counting bandwidth and storage costs and including only the compute cost for decoding as assumed above, the cost of reading data may be significantly less than that required for bit-by-bit sequencing processes. This cost may be further reduced if all data storage and computing happens locally, rather than in the cloud.

FIG. 32 shows a system diagram of archival operations. The archive (CAR) is partitioned into boot, ontology, index, and content regions. The boot partition may be written using a standard encoding scheme capable of being decoded without any external metadata, and may store the parameters, keys, and addresses needed to read the other partitions. The OS abstracts the storage medium as a collection of fixed size blocks, as described above. Each block is a contiguous sequence of identifier sequences in a single identifier library, stored as a single pool of identifiers. (A block may, however, be mirrored in several pools for fault tolerance.) The OS can be responsible for organizing, allocating, and writing blocks. When the block layer receives a source bit stream packet, the block index divides and allocates the packet to blocks in the archive. The boot partition comprises the block index, a hierarchical data structure mapping block IDs to physical addresses (comprised of container and identifiers). The block index tracks free/used blocks and allocate blocks to new packets. Each block ID can be a logical address and can be converted to a physical address in the molecular archive. This is achieved by traversing the Block Index illustrated in FIG. 32. Each node in the data structure, called an address node, contains a sequence of child block id ranges, similar to a B-Tree data structure (e.g., as described below in relation to FIG. 34). Each range points to the next block on the path to the block of interest. In this way, a tree of address nodes is maintained culminating in leaf nodes pointing to blocks in the molecular archive which contain actual data. The leaf node so reached holds the block ID identifying, the physical address of the block, and can also hold the hash of the block. Internal nodes can also contain a hash the concatenation of the hashes of its child nodes, thus forming a hash tree. The physical address of a block comprises a Location Code, a Container Code, and an identifier range defined by a start and end identifier that described a plurality of identifiers. A block ID may resolve to more than one physical address to enable fault tolerance. For example, it may be spread across two or more disparate containers, or two or more disparate identifier ranges.

At the molecular level, retrieval queries are answered using a combination of two operations: an identifier sub-library selection operation (for example, with the nested PCR or nested affinity tag access methods described above) and an identifier reading operation (for example, with the impedance-based methods described above, or with sequencing-by-synthesis). Each operation has a positive cost and takes a positive amount of time, measured in minutes to hours. A selection operation may involve a number of sequential PCRs that recursively select identifiers with a given fixed prefix of components. For an LCPCC with L layers with each component sequence of length l_{c} bases, performing p sequential PCRs (SPCRs) on an identifier library will decrease the identifier library diversity by Op layers, where θ is the number of layers covered by a single PCR. Thus, after p SPCRs on non-multiplex layers, the identifiers present are diverse in L−θp layers. Each such identifier corresponds to (L−θp)l_{c} diverse bases. If a sequencing technique is capable of reading molecules of maximum length 6, then the number p of SPCRs needed to be able to sequence any identifier after p SPCRs is constrained in the following way:

$$p \geq \frac{1}{θ} ( L - \begin{matrix}σ \\ {l}_{c}\end{matrix} ) .$$

After p SPCRs where p satisfies the given constraint, an identifier library of span D possible identifiers is truncated to an identifier library containing D′ identifiers, where

$${D}^{′} = \frac{D}{{c}_{b}^{{θ}_{p}}} ,$$

where c_{b} is the number of components in any base layer of the partition scheme. Assuming an encoding scheme with L=15 layers and c_{b}=6, l_{c}=30 bases, σ=300, and θ=4, gives p=2. In this case, the truncated identifier library size D′≅6×10⁵≤10⁶ identifiers. For example, assuming a simple model of perfectly uniform enrichment of each identifier, the sample size that may be required to sample all identifiers with high probability may be calculated using a Coupon Collector model, and turns out to be S≥βD′ ln D′, where

$$β = 1 - \frac{In α}{In {D}^{′}} ,$$

and α is the probability of an incomplete sample. Setting α=10⁻⁶, gives β=2, and approximately S≥28×10⁶, showing that a sample size of 28×the size of the selected sub-library may be sufficient. Note that the uniform enrichment assumption is idealistic and the coverage may need to be somewhat larger (but not extraordinarily so, given that non-uniform coupon collection distributions are also known to be concentrated around the mean). This allows a tolerance of a 10- to 100-fold higher value of a.

The number of block operations needed may be calculated via the following steps. As an example, consider data blocks of size 10⁶ bits in an archive of 10¹⁹ bits, roughly an Exabyte. The archive is composed of 10¹³ data blocks partitioned into 10⁷ compartmentalized identifier libraries, each containing 10¹² bits. Each identifier library contains 10⁶ data blocks. If each blocks is represented by a continuous range of ordered identifiers, then each block may be completely defined by the sequence of components in its first identifier and that in its last identifier. For an L-layered scheme, with a component library of C components, a physical block address comprising an identifier range can thus be encoded using 2L[log₂ C] bits. Thus, if C=112 and L=15, then the identifier range may be encoded in 27 bytes. By similar reasoning, encoding a physical container address may require at least 3 bytes. A total of 64 bytes may be allocated for encoding a physical block address comprising an identifier range and a physical container address. We can allocate 128 bytes to store a hash. Analogously, a source block ID for any block in the source bit stream may require 64 bytes. An address node that can support up to 512 child nodes may require 511 block ID range markers and 512 pointers, each of which is a physical archive address (for example, container address and identifier range) of a child block. Thus, an address node may require 511×64+512×64+128 bytes, or 524,800 bits. Including error correction bytes, such an address node would be encodable in a 10⁶-bit archive block. Thus, a 10⁶-bit archive block could be selected and read.

Using such a scheme, a hierarchical block addressing structure for an Exabyte-scale archive would need to be no more than five levels deep. Starting from a “cold” cache of blocks, i.e., no blocks have been queried, a single-block access with a block ID may require at most six select-read operations: five select-read pairs to find the physical address of the block in question (e.g. traversing through the B-tree), and one select-read pair to read the data block. The OS can use this foundation of a block and a block index to build an indexable archive. The basic unit of storage is a block and blocks refer to other blocks using block ids which are mapped to physical addresses using the block index.

Each high level operation on a block of bits depends on and results in a number of physical operations, which rely on chemical methods or physical steps to be orchestrated. This can involve two types of software tools: optimization tools that translate block operations into an optimized set of physical operations, and translation tools that convert physical operations into detailed programs of actions to be executed by technicians or automation devices, and may include designing and implementing a translator between operations on blocks of bits and physical and chemical operations.

The OS can also allocate bottleneck resources—liquid handling robot and technician time for example—and schedule other write operations so as to minimize the make-span of a write operation. A read request may arise from a pattern query (as described below) and comprise reading a range of blocks specified by block IDs. Because a CAR index is organized as a B-Tree (e.g., as described below in relation to FIG. 34), each pattern query is likely to translate into an access of a range of adjacent words in a range of adjacent blocks.

Typically, pattern queries involve conjunctions and disjunctions and are translated into an optimized sequence of join operations to minimize the read footprint. The query optimizer used in the systems and methods described herein may be modified and augmented so as to faithfully reflect the access cost model in a CAR. After join optimization, a complex pattern query comprising conjunctions and disjunctions may result in a set of blocks to be read. The OS may generate chemical steps for reading the block index to convert block IDs into physical addresses (container and identifiers) and the steps for reading the target blocks. Reading a set of target blocks may require identifying the set of primers needed to execute a set of sequential PCRs as identified by the query optimizer. The OS can generate instances of the SPCR and other chemical methods, complete them with physical sample addresses and method parameter values like volume and concentration, and allocate all essential labware, instruments, and technicians. The overall framework can be responsible for three tasks: time scheduling, resource allocation, and compilation and generation of action programs. The OS can adapt extant frameworks from the business process modeling and automation space such as jBPM and Camunda and from cloud workflow tools such as Simple Workflow Service and Logic Apps may be adapted for the framework.

The OS can interact with at least four automation devices: the writing system, the reading system, a liquid handling robot, and a sample and container management system. The OS can translate the steps in the workflows output by the physical and chemical translators automatable by any of these devices into device-specific instructions. The writing system can be programmed as to which sets of components to collocate, and may require the design and generation of a reaction specification format. FIG. 19 illustrates an example format, and shows how a combinatorial space may be used to encode a bit stream and serialized into a reaction specification. In this example, a 5-bit codeword of fixed weight three bits encodes a source word. The codeword is translated into a combinatorial space of four base layers and a multiplex layer comprising five components, corresponding to the bits in a codeword. Each codeword is to be assembled in a single reaction, and each reaction may include 4 base components and 3-of-5 multiplex layer components, or 7 components in total. A combinatorial space from a layered product constructor can be visualized as a trie and each path from root to leaf in this trie is an identifier. The set of identifiers to be assembled in each reaction can be serialized by traversing the trie in post order, as depicted at the bottom. This reaction set specification is generated by the OS for each write job and packaged with additional description information in the form of job and block descriptors, illustrated in FIG. 33.

FIG. 33 shows a writing workflow with a printer-finisher system, according to certain illustrative embodiments. Print job instructions are split into print head controller command frames and sent to individual print heads, each capable of printing up to four inks, each ink containing a single component. The writing system is equipped with cameras imaging nozzle test (chirp) signals. The OS will process this image data offline, estimate a write error rate, and generate rewrite requests for potentially missing identifiers until the error rate becomes tractable by error protection scheme. The OS will also produce instructions for directing a container management system to submit and checkout containers, and generate container barcode labels. For an Exabyte-scale archive, up to 10⁶ identifier libraries can be managed by a container bank such as the Hamilton Verso or the Brooks SampleStore II. Such container banks also integrate with third party laboratory information management systems (LIMS): for example, the Verso API is supported by LabVantage 8 LIMS and Mosaic. The Verso and Mosaic both support programmatic control through RESTful APIs for integration with the OS. The OS may also generate “pick-list” transfer instructions for liquid handlers to facilitate sample prep for sequencing and SPCRs.

The systems and methods described herein provide preservation, discovery, and querying of an archive incrementally, without requiring the decoding large portions of the archive. It should be possible to discovery, query, and read target content selectively and incrementally, while minimizing the need to compute joins and other structures on the archive. A key metric to be minimized is the total number of bits decoded to satisfy a sequence of queries.

FIG. 34 shows an example of a data path for the writing system. Semantic annotation of data blocks may be provided and comprise a block ID, with relational information linking them, and a compact index mapping terms to blocks for access. A Resource Description Framework (RDF) may be as the framework implementing the discovery layer to address indexing and discoverability while minimizing cost of access and maximizing longevity of interpretation. RDF is a formal meta-language standardized by the World Wide Web Consortium for representing information about resources as a set of subject-predicate-object (SPO) triples encoding a graph of entities. Key advantages of RDF can be leveraged, including the ability to interlink blocks of data and even datasets, the integration of domain-specific vocabularies, and tooling for translation, indexing, compaction, and mining.

An archive can be factored into four partitions: bootup, ontology, index, and content. The ontology partition contains a vocabulary of terms and classes and a list of the labels of all entities found in the archive. As such, it offers a taxonomy of the contents of the archive and is intended to facilitate targeted discovery while minimizing the decode footprint. The ontology may be provided by the producer or constructed during ingest using domain-specific software. The index partition can be organized as a triple-store. For example, it can store several collections of lexicographically sorted triples—typically between 6 and 18—with the goal of rapidly serving pattern queries. RDF querying is by example and takes the form “select x₁, x₂, . . . xₖ where {p₁ and p₂ and . . . pₙ}”, where xi are data attributes of interest and pi are subject-predicate-object patterns, where each is either a variable or a literal value. For example, “select name where [author <hasName> name and book <writtenBy> author and movie <basedOn> book and movie <directedBy> dir and dir <hasName>“Kubrick”}” returns the names of all authors whose books were made into films directed by “Kubrick.” Because each element of a pattern may be a literal or a variable, any of the six permutations of SPO may be needed to search for triples satisfying a pattern in a query. Additional indices storing all unary and binary relations (e.g. SO, SP, OS, etc.) with a count of satisfying triples may also be maintained. These indices are stored as B-Trees or as tries (as described below in relation to FIG. 34), where literals in a pattern serve as the key through the B-Tree or trie and the satisfying triples are stored in blocks pointed to by leaf nodes. The RDF Header Dictionary Triples framework may be used to implement this index organization and serialization scheme. While RDF literals may be string URIs, these are mapped to integer ids when stored in these indices. Because adjacent triples may share prefix literals, they can be stored as deltas in a compressed form and may be directly translated into ranges of identifiers.

An advantage of such a native semantic data storage is that a query may now be satisfied without having to read and decode the entire index. An analyst-initiating discovery of an archive results in the decoding of the bootup and ontology partitions. These are disseminated to the analyst from which the analyst may construct initial queries. A RDF query engine (e.g. Redland Rasqal), coupled with the software described herein, can transform this pattern query into an optimized set of scans through the B-Tree indices, and CAR blocks. The result may be a set of satisfying triples, and/or a set of estimated triple counts. Based on these results, the analyst may submit further queries that may result in decoding of long-form target content comprising images, videos, or scientific datasets. Because indexing is natively integrated with Applicant storage, the decoded footprint can be minimized by the query optimizer.

Each entity included in the index is referred to by an integer ID, which expands to a Uniform Resource Identifier (URI). The entity referred to by the URI, however, is stored in the content partition, which may optionally have an additional compressed index. Compressed indices (e.g. a wavelet tree) enable multi-resolution access, multi-scheme writing, and fast searching, all with small space overheads.

FIG. 34 shows a flowchart 1800 outlining the steps for storing blocks of data associated with block identifications (IDs) in containers. At step 1802, a plurality of blocks is obtained. Each block comprises a string of symbols and is associated with a block ID. A block ID may be any identifying characteristic or symbol associated with a particular block. For example, it may be a semantic annotation in the form of a triple. In some implementations, a block ID is an integer, a string, a triple, a list of attributes, or a semantic annotation. For example, the first X symbols of a string of symbols included in the block may indicate a numerical ID for that block.

At step 1804, a block (one of the blocks belonging to the plurality of blocks received in step 1802) is assigned to a container. A container may be a physical location, such as a bin, tube, or other physical storage medium where nucleic acid molecules may be stored. A container may be linked to a single block or multiple blocks. For example, one container may be associated with B blocks of information. In some embodiments, a container may comprise multiple sub-containers.

At step 1806, the block is mapped to identifier sequences to be associated with the container. These identifiers may comprise an identifier range or multiple disparate identifiers of identifier ranges. An identifier range may be specified by the component sequences that comprise the identifiers flanking the range. In some implementations, each individual identifier is associated with a distinct integer, such that an identifier range may be specified by two integers. An individual identifier sequence of the plurality of identifier sequences corresponds to an individual symbol in the string of symbols stored in the block. Each identifier sequence includes a corresponding plurality of component sequences. Each of these component sequences includes a distinct nucleic acid sequence.

At step 1808, individual identifiers of the plurality of identifier sequences are constructed. For example, a set of Q identifier sequences may be associated with a particular container. A subset V of those Q identifier sequences may be physically constructed to represent information in the block, as described in various methods described above.

At step 1810, the identifiers constructed in step 1808 are stored in the assigned container. For example, the assigned container may then hold a number V of identifiers representing the information stored in the block. Identities of the container and the plurality of identifier nucleic acid sequences associated therewith are configured to be determined using the associated block ID. In some implementations, the identities are stored in a data structure designed to facilitate access of the identity of each container using the associated block ID. For example, the data structure may be one of a B-tree, a trie, or an array. In some implementations, at least a portion of the data structure is stored along with the digital information in an index. The index may include a second plurality of identifier sequences associated with a second container. In some implementations, the index is stored in a magnetic storage device, an optical storage device, a flash memory device, or cloud storage

If the index includes a B-tree data structure, each node of the B-tree may include a distinct plurality of identifiers (i.e., different than the set of identifiers constructed in step 1808) of the second plurality of identifier sequences. In some implementations, to determine the identity of the distinct plurality of identifiers, the B-tree may be searched. Searching for a particular block ID in the B-tree may include selecting the distinct plurality of identifiers that comprise a first node and reading a value of the first node. The steps of selecting an identifier and reading a value of a node may be repeated with subsequent nodes. The identity of the distinct plurality of identifiers that comprise the subsequent node is determined by the block ID in relation to the value of the first node. In an example, the first node is the root node of the B-tree and the process of selecting (with random access method described above) and reading nodes continues until the value of a leaf node of the B-tree is read. The value of the leaf node is configured to communicate whether the block for the block ID exists. If the block ID exists, the identity of the container and the identity of the plurality of identifier nucleic acid sequences comprising said block (for example, the identifier range) may be communicated to a user or system.

If the index comprises a trie data structure, each node of the trie may comprise a distinct plurality of identifiers of the second plurality of identifier sequences. In some implementations, the block ID is a string of symbols and each node in the trie corresponds to a possible prefix of the string of symbols. If a path through the trie for a block ID exists, then the physical address (comprised of the container and identifier range or ranges) of the corresponding block can be specified by the leaf node of that path. Each intermediate node of the trie can be represented by a separate plurality of identifiers and can contain information on how many daughter nodes it has, what symbols those daughter nodes represent, and the physical addresses (comprised of the container identity and identifier range or identifier ranges) of those daughter nodes. In that way, the trie can be navigated in DNA, similar to the B-tree, using select-read operations as described above.

If the data structure is an array, each element of the array comprises a distinct plurality of identifiers of the second plurality of identifier sequences. In some implementations, each element in the array corresponds to a block ID and each element contains the physical address (comprised of the container identity and identifier range of ranges) of that block ID.

In some implementations, the location in the index of the identities (of the container and plurality of identifiers for each block) introduced in step 1810 described above is natively configured to the block ID. The block ID may map directly to a plurality of components that only and all identifiers that contain the physical address of the block share. The plurality of identifier nucleic acid molecules in the index that stores the identities may be comprised of individual identifier nucleic acid molecules that each comprise the plurality of components. In some implementations, the block ID maps directly to the container and a plurality of nucleic acid components that only and all identifiers of the corresponding block in the container share. In some implementations, the block ID is a triple of entities that annotate the associated block, and an entity of the triple maps to a plurality of nucleic acid components. The plurality of identifiers in the index that comprise individual identifiers that comprise the components can store the physical addresses of all blocks annotated with the entity. Alternatively or in addition, the plurality of identifiers in the content of an archive that comprise all blocks annotated by said entity may exclusively comprise the corresponding plurality of nucleic acid components that represent said entity.

A system for storing digital information according to any of the methods described herein (e.g., the methods described in relation to FIGS. 24-26, 34) may include a sample management system for storing multiple containers of nucleic acids. The system may use automation machinery for retrieving a specified container from the sample management system. In some implementations, this sample management system is used to access any kind of containers—including the DNA-holding identifiers described above in relation to FIG. 34 but also including any kind of physical or computer represented data storage element.

The foregoing is merely illustrative of the principles of the disclosure, and the apparatuses can be practiced by other than the described embodiments, which are presented for purposes of illustration and not of limitation. It is to be understood that the methods disclosed herein, while shown for use in nucleic acid-based data storage, may be applied to systems in other applications requiring data storage.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

The systems and methods described may be implemented locally on a printer/finisher system. The printer/finisher system may include a data processing apparatus. The systems and methods described herein may be implemented remotely on a separate data processing apparatus. The separate data processing apparatus may be connected directly or indirectly to the printer/finisher system through cloud applications. The printer/finisher system may communicate with the separate data processing apparatus in real-time (or near real-time).

In general, embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a computer readable medium for execution by, or to control the operation of, data processing apparatus. The computer readable medium can be a machine-readable storage device, a machine-readable storage substrate, a memory device, a composition of matter affecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program may correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices.

Examples of changes, substitutions, and alterations are ascertainable by one skilled in the art and could be made without departing from the scope of the information disclosed herein. All references cited herein are incorporated by reference in their entirety and made part of this application.

What is claimed is:

1. A method for writing digital information into nucleic acid molecules, the method comprising:
mapping the digital information to a target set of identifier nucleic acid sequences;
obtaining a plurality of identifier nucleic acid molecules;
sequencing an identifier nucleic acid molecule of said plurality of identifier nucleic acid molecules with a nanopore system; and
accepting or rejecting the identifier nucleic acid molecule into a destination chamber based on whether or not the identifier nucleic acid molecule corresponds to an identifier nucleic acid sequence of the target set.

2. The method of claim 1, wherein said mapping comprises using a codebook that maps a word to a codeword.

3. The method of claim 2, wherein at least one identifier nucleic acid sequence corresponds to a bit in the codeword.

4. The method of any of claims 1-3, wherein if said bit has a bit-value of 1, said bit is represented by a presence of the at least one corresponding identifier nucleic acid sequence in the target set, and if the bit has a bit-value of 0, said bit is represented by an absence of any corresponding identifier nucleic acid sequences in the target set.

5. The method of any of claims 1-4, wherein said plurality of identifier nucleic acid molecules is obtained by assembling multiple component nucleic acid molecules using a product scheme, wherein the product scheme defines a set of M layers, each layer comprising a set of components, and wherein each identifier nucleic acid molecule contains one component from each layer of the set of M layers.

6. The method of any of claims 1-5, wherein said plurality of identifier nucleic acid molecules is obtained by programmably synthesizing multiple oligonucleotides with de novo synthesis.

7. The method of any of claims 1-6, wherein said plurality of identifier nucleic acid molecules is obtained by synthesizing degenerate oligonucleotide sequences.

8. The method of any of claims 5-7, further comprising incorporating common primer binding sites to each identifier molecule of the plurality of identifier nucleic acid molecules.

9. The method of claim 8, further comprising amplifying the plurality of identifier nucleic acid molecules with polymerase chain reaction (PCR) using PCR primers configured to bind to said common primer sites.

10. The method of any of claims 5-7, further comprising adding a spacer sequence to each identifier nucleic acid molecule of the plurality of identifier nucleic acid molecules.

11. The method of claim 10, wherein the spacer sequence is added by one of ligation or overlap extension PCR.

12. The method of claim 11, wherein the spacer sequence is inserted into a target insertion site within the identifier nucleic acid sequence.

13. The method of any of claims 10-12, wherein the spacer sequence is configured to increase a translocation time of each identifier nucleic acid molecule of the plurality of identifier nucleic acid molecules during sequencing in the nanopore system.

14. The method of any of claims 1-13, wherein the nanopore system comprises a source chamber, a membrane, a nanopore, and the destination chamber.

15. The method of claim 14, wherein accepting the identifier nucleic acid molecule comprises translocating the identifier nucleic acid molecule from the source chamber to the destination chamber through the nanopore in the membrane.

16. The method of claim 15, wherein sequencing the identifier nucleic acid molecule comprises detecting an impedance signal and matching the impedance signal to one of multiple impedance signatures.

17. The method of claim 16, further comprising binding an agent to each identifier nucleic acid molecule of at least a subset of the plurality of identifier nucleic acid molecules to provide a distinct impedance signal.

18. The method of claim 17, wherein the binding comprises binding the agent to each identifier nucleic acid molecule of the plurality of identifier nucleic acid molecules.

19. The method of any of claims 16-18, wherein the identifier nucleic acid molecule is accepted or rejected into the destination chamber based on at least one impedance signature to which the identifier nucleic acid molecule matches.

20. The method of any of claims 1-19, wherein rejecting the identifier nucleic acid molecules comprises reversing a polarity of an electric field across the nanopore.

21. The method of any of claims 1-20, further comprising sequencing multiple identifier nucleic acid molecules in the nanopore system until the destination chamber comprises a plurality of identifier nucleic acid molecules that is sufficient for representing the digital information with error correction.

22. The method of any of claims 1-21, wherein mapping comprises using forward error correction.

23. The method of any of claims 1-22, further comprising correcting for any errors that occur during the sequencing step or the accepting or rejecting step by using backward error correction.

24. The method of any of claims 1-23, wherein the destination chamber is a first destination chamber, and the target set is a first target set, and wherein the method further comprises:
accepting or rejecting the identifier nucleic acid molecule into a second destination chamber based on whether or not the identifier nucleic acid molecule corresponds to an identifier nucleic acid sequence of a second target set.

25. The method of claim 24, wherein the nanopore system comprises a source chamber, a first membrane, a first nanopore in the first membrane, a second membrane, and a second nanopore in the second membrane; and
wherein the first membrane separates the source chamber and the first destination chamber, and the second membrane separates the source chamber and the second destination chamber.

26. The method of any of claim 24 or 25, wherein if said bit has a bit-value of 1, said bit is represented by a presence of the at least one corresponding identifier nucleic acid sequence in the first target set, and if the bit has a bit-value of 0, said bit is represented by a presence of the at least one corresponding identifier nucleic acid sequence in the second target set.

27. The method of any of claims 24-26, further comprising:
designating a probe set of component nucleic acid sequences;
sequencing a probed identifier nucleic acid molecule from the first destination chamber or the second destination chamber with the nanopore system; and
accepting or rejecting the probed identifier nucleic acid molecule into a retrieval chamber based on whether or not the probed identifier nucleic acid molecule corresponds to an identifier nucleic acid sequence containing a component nucleic acid sequence of the probe set.

28. The method of any of claims 1-27, wherein accepting or rejecting the identifier nucleic acid molecule comprises:
accepting the identifier nucleic acid molecule into the destination chamber if the identifier nucleic acid molecule has an identifier nucleic acid sequence of the target set; and
rejecting the identifier nucleic acid molecule from the destination chamber if the identifier nucleic acid molecule does not have an identifier nucleic acid sequence of the target set.
